(19) United States
(12) Patent Application Publication
(10) Pub. No.: US 2024/0110101 A1
(43) Pub. Date: Apr. 4, 2024

(54) Ferroelectric liquid crystal (FLC) material and chiral compound

(71) Applicants: VALERII VLADIMIROVICH VASHCHENKO, Hong Kong (HK); SANMA ELETRON (HONG KONG) APPLIANCE COMPANY LIMITED, Hong Kong (HK)

(72) Inventors: VALERII VLADIMIROVICH VASHCHENKO, Hong Kong (HK); OLENA V. VASHCHENKO, Hong Kong (HK); ABHISHEK KUMAR SRIVASTAVA, Hong Kong (HK)

(21) Appl. No.: 18/504,269

(22) Filed: Nov. 8, 2023

Related U.S. Application Data
(60) Provisional application No. 63/423,647, filed on Nov. 8, 2022.

(51) Int. Cl. C09K 19/58; C07C 69/78; C07D 213/80; C09K 19/02; C09K 19/34

(52) U.S. Cl. CPC C09K 19/588; C07C 69/78; C07D 213/80; C09K 19/0225; C09K 19/3461; C09K 19/586

(57) ABSTRACT

A ferroelectric liquid crystal (FLC) material in a vertically aligned liquid crystal cell, comprising at least one chiral compound and at least one non-chiral liquid crystal compound, with a sub-micron scale helix pitch less than 300 nm, spontaneous polarization greater than 100 nC/cm2, and a tilt angle greater than 38 degrees. The ferroelectric liquid crystal (FLC) material provides an extremely large Kerr constant in a vertically aligned liquid crystal cell at the communication band of the light and therefore reduces the driving voltage and response time.

### CROSS-REFERENCE TO RELATED APPLICATIONS

This application claims priority benefits to US provisional Application No. 63423647 filed Nov. 8, 2022, the content of which is incorporated herein by reference.

### TECHNICAL FIELD

The present invention relates to ferroelectric liquid crystal (FLC) materials, and in particular, to deformed helix FLC (DHFLC) type of FLC materials.

### BACKGROUND

Fast, high-resolution, and continuous >2π phase modulation is in high demand for a wide and diverse range of applications. These include photonic devices such as tunable lenses, wave front correctors, focusers, and correlators. The state-of-art near-to-eye displays meet barriers in realization of >2000 pixel density (PPI) and fast frame rate >360 Hz. Regarding existing limitations, modern LC devices suffer from low contrast (<1000:1), small phase depth (<2π), slow response (˜8 ms), non-conserved ellipticity and hysteresis. The fast phase modulation is also important for the field sequential color displays, where the power consumption can be reduced by at least 3 times.

Kerr-effect is a promising electrooptical (EO) effect for fast phase modulations. In Kerr-effect based devices, the birefringence (Δn) shows quadratic voltage dependence given by:

Δn=n_{E∥}−n_{E⊥}λKₖₑᵣᵣE² (1)

where λ is a wavelength of incident light, E is an applied electric field and Kₖₑᵣᵣ is Kerr constant. The n_{E∥} and n_{E⊥} are refractive index parallel and perpendicular to the molecular induced by electric field.

Compared with other EO effects, a non-linear relationship between electrical field and induced birefringence in Kerr-effect offers two vital advantages. First, achieving same phase depth within smaller voltage and shorter time; Second, common liquid crystal systems maintain optical anisotropy regardless of whether an external field is applied or not. Various materials and methods are proposed in the past decades. Among them, (i) polymer-stabilized blue phase liquid crystal (PSBPLC), cholesteric liquid crystal (CLC), and (iii) ferroelectric liquid crystal (FLC) in a deformed helix mode (DHFLC) are best potential candidates for Kerr-effect-based phase modulation.

The PSBPLC, in absence of an external electrical field, shows quasi-optical isotropy (n_{∥}=n_{⊥}) due to high symmetry in a structure of their cubic lattices, the refractive index ellipsoid is spherical. When the external electrical field is applied, the symmetry of the lattice distorts, the refractive index in 3D changes from a sphere to an ellipsoidal (n_{∥}>N_{⊥}), thus induces a birefringence. An additional advantage is that no surface treatment is required in a fabrication process. However, a dark state needs further improvement and high operation voltage, tens of V/μm, is essential for a low-hysteresis mode.

The CLC, in the absence of the electric field, has a helical structure, which is undistorted with non-transmissive device between crossed polarizers. When an in-plane electric field is applied, an optic axis of the CLC rotates, inducing a birefringence. Apart from that, the device becomes transmissive for a sufficiently large induced tilt angle (a required angle depends on a birefringence of the LC). The short-pitch CLC (pitch (p0)<<wavelength (λ)) is considered to be uniaxially birefringent structure in a USH (uniformly standing helix) mode with the optic axis along a helical axis. The pro about CLC is contrast ratio >3000:1, but high electric field (E), high hysteresis and just π phase depth are currently achieved, thus further optimization is required.

Regarding FLCs, most of their modes such as SSFLC, ESH are not suitable for continuous phase modulation devices because FLC optical axis sweeps in the plane of the cell substrate producing changes in the polarization state of the incident light. Only suitable is DHFLC that in vertical alignment (VADHFLC) shows highest K_{Kerr} and fastest switching time at lowest driving voltage, see Table 1.

TABLE 1
Comparison of Kerr-effect Based LC Configurations in the past

| Tech | Kₖₑᵣᵣ, nm/V² | λ, nm | Ε, V/μm | τ(μs) | Hysteresis, % |
|---|---|---|---|---|---|
| PSBPLC | 33 | 514 | 10-20 | 500 | 0.36 |
| ChLC | 0.187 | | 20 | 400 | ±5 |
| VADHFLC | 27 | 543 | | 100 | no |
| | 104.6 | 450 | ~2 | 300 | no |
| | 85.1 | 543 | | | |

Although parameters of the VADHFLC are the best comparatively to other technologies, see Table 1, they are still not enough for practical application and should be further upgraded. Particularly, the key parameter defining the light modulator performance, the Kerr constant (K_{Kerr}) was still small that should also be improved to increase the phase depth and reduce the driving voltage.

### BRIEF DESCRIPTION OF THE DRAWINGS

Other purposes and features described above of the present disclosure will become clearer by the following description in conjunction with the attached drawings.

FIG. 1 is a phase diagramme of two-ring compounds mixture PP-10 with PyP-4, where the vertical dotted line denotes the eutectic composition, a mixture PPy-1, at PP-10 content of 46.4%.

FIG. 2(a) is a phase diagramme of a mixture BPP-4 with PP-10.

FIG. 2(b) is a phase diagramme of a mixture BPP-4 with PPy-1.

FIG. 3(a) shows temperature dependency of helix pitch for chiral dopants S-DFT-TFA-6 and S-PDN-TFA-6.

FIG. 3(b) shows temperature dependency of HTP (Helical Twisting Power) for chiral dopants S-DFT-TFA-6 and S-PDN-TFA-6.

FIG. 4(a) shows temperature dependency of tilt angle for the chiral dopants S-DFT-TFA-6 and S-PDN-TFA-6 in BPP-4 host.

FIG. 4(b) shows temperature dependency of spontaneous polarization for the chiral dopants S-DFT-TFA-6 and S-PDN-TFA-6 in BPP-4 host, where the dotted line is approximation except 3 points at temperatures 20, 30 and 40° C.

FIG. 5(a) shows dependency of transmittance at 632 nm vs voltage of FLC-9-087.

FIG. 5(b) shows dependency of transmittance at 632 nm vs voltage of FLC-146.

FIG. 6(a) shows temperature dependency of tilt angle for FLC mixtures comprising

binary chiral dopants: S-Lact-n with S-PDN-TFA-6.

FIG. 6(b) shows temperature dependency of tilt angle for FLC mixtures comprising binary chiral dopants: S-Lact-n with S-DFT-TFA-6, where single-chiral dopant mixture 9-146 is given as a reference.

FIG. 7(a) shows temperature dependency of spontaneous polarization for the FLC mixtures comprising binary chiral dopants: S-Lact-n with S-PDN-TFA-6, where single-chiral dopant mixture 9-146 is given as a reference.

FIG. 7(b) shows temperature dependency of spontaneous polarization for the FLC mixtures comprising binary chiral dopants: S-Lact-n with S-DFT-TFA-6.

FIGS. 8(a) shows dependencies of transmittance at 632 nm vs voltage of mixtures comprising binary chiral dopants S-PDN-TFA-6/S-Lact-4 9-147.

FIGS. 8(b) shows dependencies of transmittance at 632 nm vs voltage of mixtures comprising binary chiral dopants S-PDN-TFA-6/S-Lact-4 9-151.

FIG. 9 shows dependencies of transmittance at 632 nm vs voltage of mixtures comprising binary chiral dopants S-DFT-TFA-6/S-Lact-4 (9-126) at different temperatures.

FIGS. 10(a), 10(b) and 10(c) shows electrooptical characteristics with temperature for FLC-9-199 mixture measured at 90 Hz, where FIG. 10(a) shows dependency of Transmittance vs Voltage, FIG. 10(b) shows dependency of Response Time_on vs voltage, and FIG. 10(c) shows dependency of Response Time_off vs voltage.

FIG. 11 shows tilt temperature dependence for mixture FLC-9-091.

FIG. 12 shows tilt temperature dependence for mixture FLC-9-196.

### DETAILED DESCRIPTION

For clear description and better understanding of the technical problem to be solved, technical solutions, and advantages of the present disclosure, the present disclosure is further described in detail with reference to the accompanying drawings and specific embodiments. It should be understood that the embodiments described here are only exemplary ones for illustrating the present disclosure, and are not intended to limit the present disclosure.

Theoretically, the K_{Kerr} depends on macroscopic DHFLC parameters as follows:

$${K}_{k e τ τ} = \frac{{n}_{p}}{λ} \frac{( {ε}_{e} - {ε}_{⊥} )}{( {ε}_{e} + {ε}_{⊥} )} \frac{{P}_{S}^{2} {p}_{0}^{4}}{32 π {K}^{2} {sin}^{4} θ} \qquad ( 1 )$$

$$where$$

$${n}_{p} = \sqrt{\frac{( {ε}_{e} + {ε}_{⊥} )}{2} ,}$$

$${ε}_{e} = \frac{{ε}_{‖} {ε}_{⊥}}{{ε}_{⊥} {sin}^{2} θ + {ε}_{‖} {cos}^{2} θ} ,$$

ε_{⊥}, and ε_{∥}, are dielectric constants measured perpendicular and parallel to a helix axis at high frequency (1 kHz), and K is an elastic constant.

Among the parameters effecting on K_{Kerr}, some of them should not be varied since they are responsible on other important properties of DHFLC, such as tilt angle θ and λ, the last is predefined by a chosen communication wavelength. Whereas the tilt angle, in turn, defines effective induced birefringence and for the best performance it should be in a range of 40-45°, actually fixing thereby the term sin⁴ θ to 0.171-0.25.

Thus, main tools for K_{Kerr} improvement remain spontaneous polarization P_{S} and helix pitch p₀. Moreover, as these parameters are taking in power 2 and 4 respectively, thus at small their changes a significant growing of K_{Kerr} should be expected. There are, however, certain limitations on variation of these parameters, which are considering below.

The p₀ value is important for several reasons.

In order to satisfy a prerequisite of DHFLC existence p₀<<cell gap, i.e., in a typically used cell gap, p₀ should be well below 1000 nm, i.e., to be in a sub micrometer range.

The pitch should be short enough to avoid Bragg diffraction of helical supramolecular structure in order to secure high contrast. The relationships between wavelength of the Bragg diffraction (λₘₐₓ) and po in the SmC* phase are expressed as follows:

λₘₐₓ=n̅p₀, (2)

Thus, for devices exposing at ambient light (eg. Displays), the helix pitch should be shifted to UV region, p₀<120 nm (referring to reference 10 below). Whereas in the case of phase modulator at communication wavelength λ=1550 nm where only normal light incidence occurs, any Bragg diffraction is not observed when p₀<1000 nm.

Short p₀ values also favors fast switching OFF time (τ_{off}) in the DHFLC given by the following equation:

$${τ}_{off} = \frac{{γ}_{φ} {p}_{0}^{2}}{2 π {K}_{φ} {sin}^{2} θ} , \qquad ( 3 )$$

where γ_{φ} is rotational viscosity, K_{φ} is elastic modulus of the helix.

The dark state of the VADHFLC strongly depends on the FLC pitch. For a good dark state, FLC should have a short-pitch, i.e. n̅p₀<<λ and so the τ_{off} was deduced as:

$${τ}_{off} = \frac{{π}^{2}}{16} \frac{Δ {n}^{4} {p}_{0}^{6} {d}^{2}}{{\overline{n}}^{4} {λ}^{8}}$$

For FLC with a high pitch τ_{off} increases that may deteriorate the optical contrast of the VADHFLC cell. This affect is most pronounced for the blue light, however, for green and red light the τ_{off} is still acceptable till pitch ˜250 nm and even to ˜400 nm at communication wavelength.

The increasing of spontaneous polarization (P_{S}) at the given FLC components required increasing the concentration of chiral component, which can increase the K_{Kerr} but simultaneously it increases γ_{ϕ} that slows down the FLC.

It is also worth noting, that light modulation should not have notable dependence on driving voltage frequency

Referring to a corresponding reference (US2023250338A1), it provides effect of molecule structure on DHFLC performance and examples of the mixtures with good enough values of key parameters (θ, P_{S}, and p₀), however, the effect on K_{Kerr} evolution was not considered there. Particularly, the tight p₀ values reported in the reference, as it follows from given above consideration, are unlikely to be expected the achievement of the optimal K_{Kerr}, which can be suitable with VADHFLC for phase modulation.

As a resuming, the trade-off between VADHFLC performance and their parameters γϕ, θ, K, P_{S}, and p₀ impose serious constraints on parameters variation and thus on increasing the K_{Kerr}. That also requires careful optimization of VADHFL mixtures compositions and selection of their components.

As stated above, each of the microscopic DHFLC parameters, K_{Kerr}, θ, p₀, P_{S}, E_{c} etc., in an ideal case should be controllable in a FLC composition independently. However, it is an essential challenge, since molecule structures of FLC components and their content are effecting on several parameters simultaneously that we are demonstrating by analysis of the following references.

WO0031582A1 provides a vertically aligned helix-deformed ferroelectric liquid crystal display, which includes: the first (10) and second glass substrates (50) each of which has two surfaces, the first (10) and second glass substrates (50) facing each other; a first transparent electrode (20) having a first potential, being formed on a first surface (12) of the first glass substrate (10); a second transparent electrode (30) having a second potential different from the first potential, being formed on the first surface (12) of the first glass substrate (10); a first vertical alignment layer (40) being formed on the first surface (12) of the first glass substrate (10), on which the first and second transparent electrodes (20, 30) are formed; a second vertical alignment layer (60) formed on a first surface (52) of the second glass substrate (50); and a ferroelectric liquid crystal (70) being filled between the first and second glass substrates (10, 50) on which the first and second vertical alignment layers (40, 60) are respectively formed, facing each other, the ferroelectric liquid crystal (70) having a shorter helix pitch than the wavelength of the light, the ferroelectric liquid crystal (70) being helix-deformed in response to an electric field applied across the first and second transparent electrodes (20, 30) so that its molecules rotate in a specific direction, thereby achieving uniform alignment and the analog gray scale capability.

In this reference, the display element exploiting actually Kerr effect is described. Sole example is given where FLC-10817 (Rolic Ltd) is used, showing P_{S} of 115 nC/cm2, tilt angle of 34° and helix pitch of 0.2 μm. This can be enough to demonstrate the potential capability of the effect in a given vertical alignment, but far from the acceptable practical application.

US2023250338A1 discloses the ferroelectric liquid crystal (FIX) materials that show an advanced performance for the electro-optical devices exploiting the deformed helix ferroelectric liquid crystal effect. The advanced performance is secured by simultaneously optimizing key parameters of the FLC material. This set of parameters is achieved by thoroughly selecting material components, as stated below:
1. The majority of the constituent material is three or four ring molecules of chiral and non-chiral components, which provides birefringence value in the range of 0.14 to 0.28 and temperature range of ferroelectric phase ranges are wider than 0 to 90° C.
2. The spontaneous polarization value of the FLC is 50 to 200 nC/cm² that is achieved by adding highly polar groups (F, CF₃, O, etc.) at the chiral centers of the chiral components. The length of the chiral components is similar to the main host components, i.e. three-ring aromatic compounds, which include the chiral units on both terminal positions.
3. The tilt angle of 35° to 46° for the FLC material is provided by matching the total length of the host with the length of chiral components molecules and by minimal differences of the terminal tail length on both sides of the central core of molecules.
4. The FLC helix pitch of 250 nm or less was provided by doping the high-twisting power chiral components in the host. The said chiral components have high polarization power and long terminal tails length, which are preferably longer than that of the host. The length of the terminal tails is optimized to meet the requirements on tilt angle stated in (c).

EP0347941A2 and EP0293763 discloses synthesis and properties of 2-(4-akylbyphenylil)-5-alkylpyrimidines.

At the beginning, the inventors of the present disclosure studied the behaviour in DHFLC mode in vertical alignment and phase modulation properties for some mixtures given in the reference US2023250338A1, which combine two chiral components with opposite sign of twisting and same signe of P_{S}. Synthesis of the chiral materials were carried out essentially as it is described in the reference US2023250338A1. Synthesis of the non-chiral compounds, which used as host, have been carried out essentially as described in references EP0347941A2 and EP0293763.

In the present disclosure, the inventors of the present disclosure developed a non-chiral liquid crystal has smectic C liquid crystal phase and is of the formula:

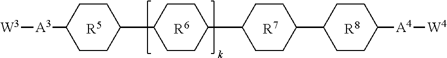

where

k is 0 or 1;

R⁵, R⁶, R⁷, and R⁸ independently are 1,4-phenylene, optionally substituted with halogen or methyl at 2 or 3 positions, or pyrimidine-2,5-diyl, or pyridine-2,5-diyl;

A³ and A⁴ are independently a single C—C bond, O, S, or an ester functional group;

W³ and W⁴ are independently alkyl CₘH₂ₘ₊₁ or alkenyl CₘH₂ₘ, where m=4-12, and optionally one or more hydrogens are independently replaced by F, furthermore, optionally one or more CH₂ are independently replaced with CF₂, O, or —CO— groups provided that two O atoms are not linked together.

In a exemplary embodiment, the inventors of the present disclosure used 6-components mixture of biphenylpyrimidines BPP-6, (see Example 5) as a non-chiral host. Mixture BPP-6 shows following phase transitions:

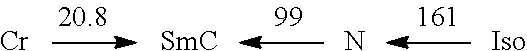

As chiral components, S-DFT-TFA6 and S-LACT-4 with formulae below in various ratios are developed. Results of studying are collected in Table 1.

| 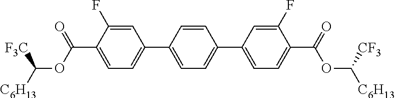 | 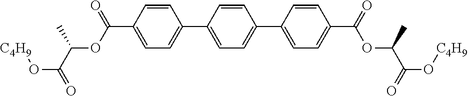 |
|---|---|
| S-DFT-TFA6 | S-LACT-4 |

TABLE 1
Main requirements to the FLC materials for application
in VADHFLC and preliminary results

| | Range of the values | | |
|---|---|---|---|
| | | Acceptable | DHFLC |
| | | variation of | mixtures |
| Parameter | Ideal case | the parameters | 9-112 |

| | | |
|---|---|---|
| Chiral comps. | S-DFT-TFA6 | 15 |
| concentrations, | S-LACT-4 | 11.2 |
| mol. % | | |

| | | | |
|---|---|---|---|
| Pitch | 50-300 | 100-300 nm | 270 |
| Cell gap/pitch (d/p) ratio | <<1 | | |
| Tilt angle θ, ° | 45 | >38 | 38.5 |
| Temperature dependence of θ | Should be | <0.2°/° C. in whole | |
| | independent | working range, | |
| Spontaneous polarization | >200 nC/cm² | >100 nC/cm² | 127.6 |
| Response time, τ_{OFF}, μs | <200 | <500 | 205 |
| Upper limit of SmC* range, ° C. | >110 | >100 | 100 |
| Storage temperature, ° C. | <−30 | <0 | >7 |
| Kerr constant, (nmV²)⁻¹ at 1550 | >250 | >100 | 51 |
| nm | | | |

Combining several types of chiral Guests being diesters of terphenyldicarboxylic acid of 1,1,1-trifluoromethyl-2-alkanols and alkyllactate in the mixtures with biphenylpyrimeidines, the inventors of the present disclosure have achieved (see Table 1):
High P_{S}.
Fully suppressing of unwanted ferroelectric phases.
Full control on induced helical pitch, which can be varied from 110 to 200 nm almost independently of other parameters.

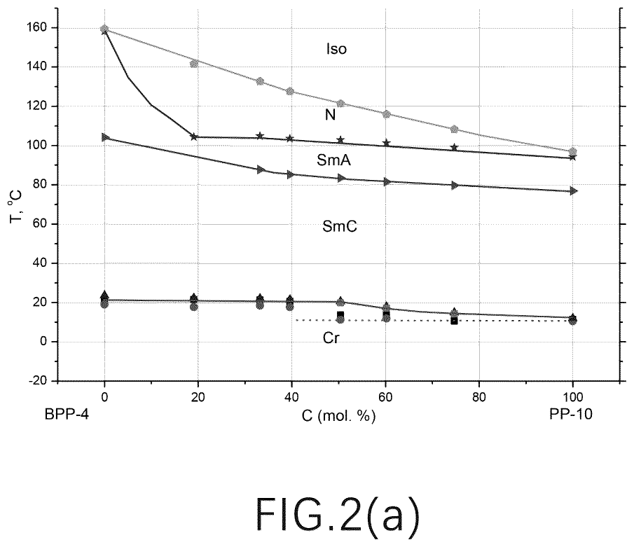

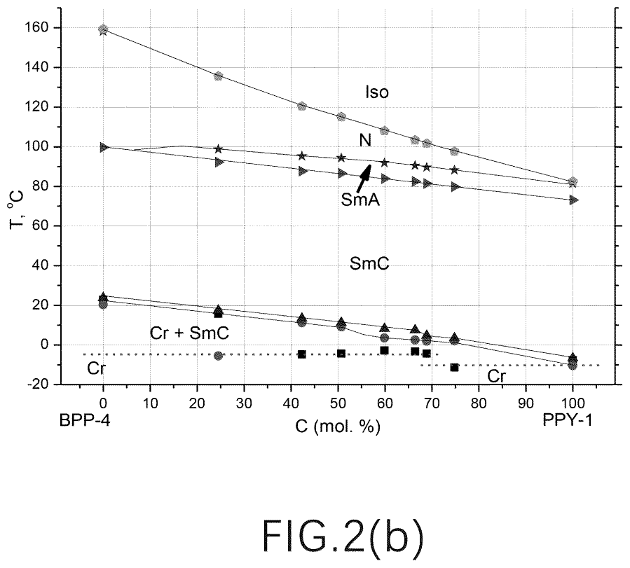

Although parameters of the elaborated mixtures (see Table 1) have closely reached to the set of the required values, they should be still upgraded. The key parameter defining the light modulator performance, the Kerr constant (K_{Kerr}) was still small that should also be improved to increase the phase depth and reduce the driving voltage. We achieved 2π phase modulation depth, which is good for the WSS (Wavelength Selective Switch) application. However, the inventors of the present disclosure found that these materials show significant frequency dependence for the electro-optical effects and has no threshold-like behavior. It is a bottleneck for application of these materials. The frequency dependence of the electro-optical effect is a result of the Goldstone mode relaxation process, which for the current set of materials, overlaps with the driving frequency of the phase modulators (see FIGS. 2A and 2B).

The Goldstone mode relaxation frequency (F_{G}) is given by

$${F}_{G} = \frac{2 K π}{γ {p}_{0}^{2}} \qquad ( 4 )$$

Thus, by reducing the pitch aiming to increase K_{Kerr}, the F_{G} is also rises up going beyond the electro-optical modulator frequency range (>1 kHz) resulting in unwanted overlaps with the driving frequency. However, reducing the pitch adversely affects the K_{Kerr}, which is proportional to p₀⁴. Thus, the idea of increasing the K_{Kerr} by increasing the pitch is not a feasible option and adversely affect the device performance. Thus, the inventors of the present disclosure need to resort on an option of further increasing P_{S} of material till simultaneous growth of viscosity will require switching time on an appropriate level.

Thus, to enhance already achieved values, following improvement are required:

| | | |
|---|---|---|
| Parameter | Desired value | Way to achieve the target values |

Phase transitions

| | | |
|---|---|---|
| melting | The should be further reduced | Re-composing of the Host. |
| point, mp | below at least 15° C. to secure FLC | Find out additional mixtures lowering |
| | upon a storage at low temperatures | melting point without affect on already |
| T_{SmC}* | The transition of SmC* to a para- | achieved parameters. |
| | electric phase (SmA, N*) should be | |
| | increased at least by 10-15 degree in | |
| | order to achieve more stable | |
| | parameters at working temperature | |
| | of 65° C. | |

Electrooptical parameters

| | | |
|---|---|---|
| θ | It should be further increased over | a) Variation of chiral component(s) |
| | 40° in order to reach higher light | structures and/or their |
| | transmission, thereby reducing the | concentration (main tool) |
| | light losses | b) Re-composing of the Host |
| P_{S} | The spontaneous polarization | (auxiliary tool) |
| | should be increase at least to values | |
| | of 200 nC/cm² | |
| F_{G} | Shifting the FG beyond the range of | Increasing the P_{S} preferably than p₀, |
| | driving frequencies, >100 Hz | the last should be kept on moderately |
| | | low level, ~200 nm |

Within the subtask about phase transitions we are taking into account that effect of reducing the melting point (mp) is weakened upon adding of each subsequent component to the mixture, i.e., one of the two FLC mixtures, containing fewer components, has potential to reduce the mp. further. In this way, instead of six-component mixture BPP-6 described in above-mentioned reference US2023250338A1, we have carefully optimized four-component mixture, BPP-4:

| | Content of the component, |
|---|---|
| Chemical formulae | mol. % |
| 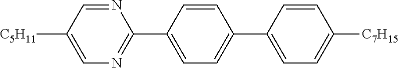 | 17.7 |
| 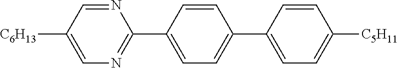 | 40.1 |
| 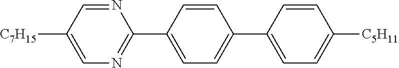 | 18.2 |
| 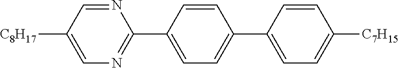 | 24 |

Mixture BPP-4 shows following phase transitions:

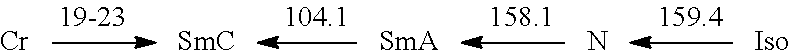

Although BPP-4 is comprised of 4 components, its melting point is almost the same as of BPP-6, whereas Tsmc is higher by 5° C.

Additionally, in order to reduce melting point of the non-chiral host, we have optimised two mixture of shorter core than BPP compounds, namely phenylpyrimidines, mixture PP-10, phenylpyridines PyP-4 and combination thereof, PPy-1.

Mixture PP-10 has following compositions:

| | Content of the |
|---|---|
| | component, |
| Chemical formulae | mol. % |
| 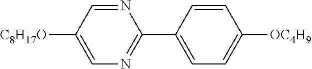 | 31.4 |
| 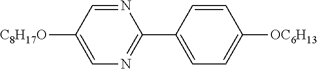 | 29.8 |
| 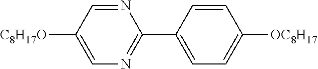 | 13.8 |
| 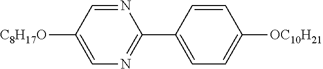 | 25.0 |

Mixture PP-10 shows following phase transitions:

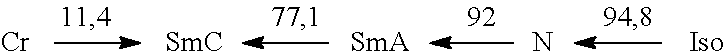

Mixture PyP-4 has following compositions:

| | Content of the |
|---|---|
| | component, |
| Chemical formulae | mol. % |
| 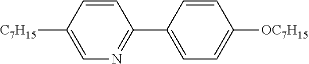 | 29.7 |
| 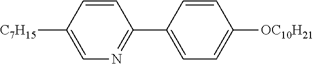 | 9.9 |
| 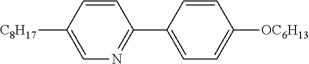 | 49.9 |
| 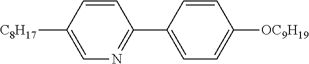 | 10.5 |

Mixture PyP-4 shows the following phase transitions:

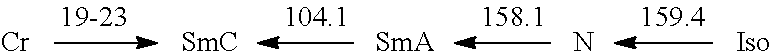

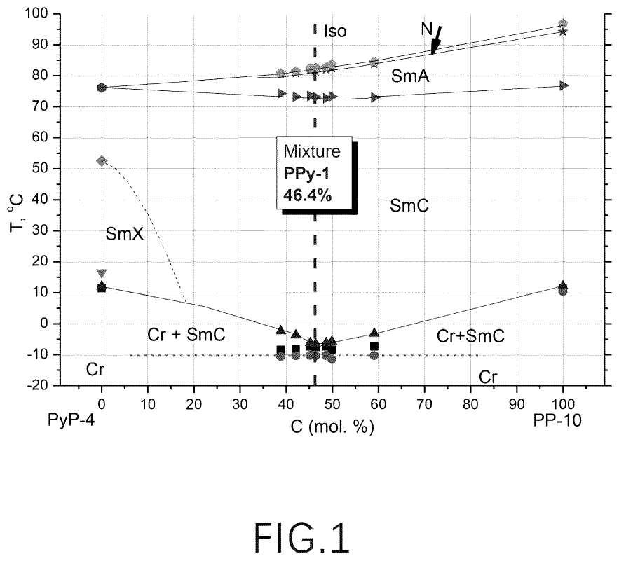

Studying of phase diagramme of PP-10 with PyP-4 allows us to develop low-melting material, PPy-1 (see FIG. 1).

FIG. 1 is a Phase diagramme of two-ring compounds mixture PP-10 with PyP-4. The vertical dotted line denotes the eutectic composition, the mixture PPy-1, at PP-10 content of 46.4%.

Mixture PPy-1 has following compositions:

| | Content of the |
|---|---|
| | component, |
| Chemical formulae | mol. % |
| 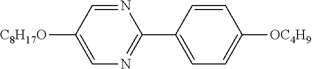 | 14.6 |
| 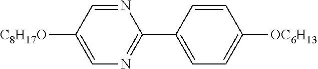 | 13.8 |
| 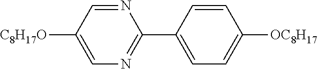 | 6.4 |
| 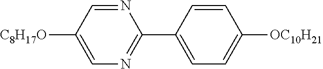 | 11.6 |
| 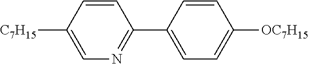 | 15.9 |
| 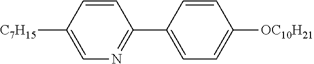 | 5.3 |
| 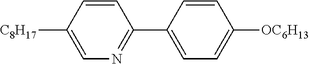 | 26.7 |
| 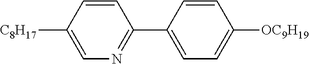 | 5.6 |

Mixture PPy-1 shows following phase transitions:

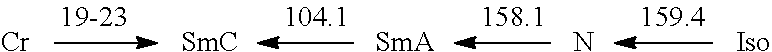

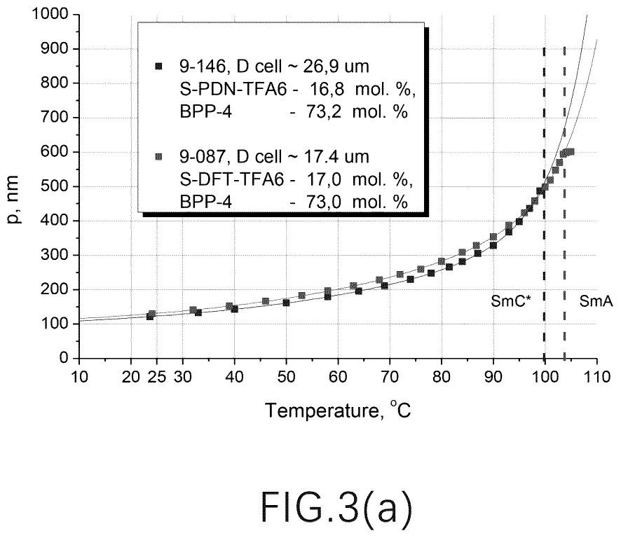

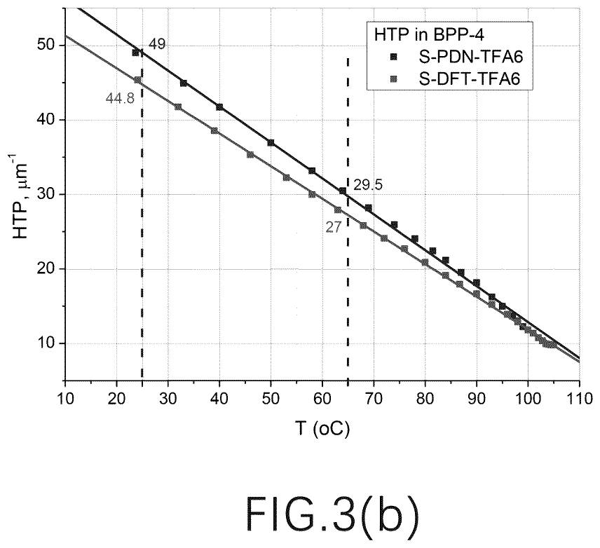

The phase diagramms of BPP-4 with new mp-reducers (mixtures PP-10, PPy-1) are shown below on the FIGS. 2 and 3. FIG. 2A is a phase diagramme of the mixture BPP-4 — PP-10, and FIG. 2B is a phase diagramme of the mixture BPP-4-PPy-1.

As it can be seen on the FIGS. 2A and 2B, the melting points indeed reduce, especially at application of PPy-1mixture. However, this effect of melting point reduction is more pronounced at high content of two-ring compounds (PPy 1), over 60% of their concentration. Moreover, the T_{SmC} is also simultaneously reduces notably, thus the mixtures based on the said two-ring compounds cannot be widely used and further optimization is required.

In order to further incrase T_{SmC} transition, we have additionally deployed with BPP-4 their O-alkyl analogues, namely the 4-akyl-4″alkyloxy-biphenylpyrimidenes, where embedding oxygen as a linker in one of the terminal alkyl results in an essentially higher T_{SmC} transition temperatures.

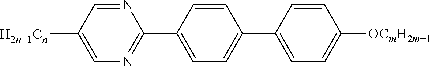

The new mixture of 6 three-ring compounds is shown below (mixtures BPPO-61-BPPO-63):

| | Content of the component, mol. % | | |
|---|---|---|---|
| Chemical formulae | BPPO-61 | BPPO-62 | BPPO-63 |
| 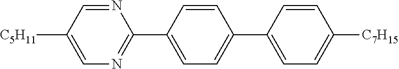 | 14.4 | 12.5 | 10.7 |
| 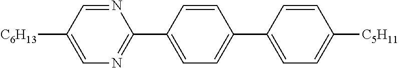 | 32.5 | 28.3 | 24.2 |
| 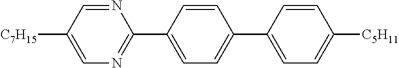 | 14.6 | 12.6 | 10.8 |
| 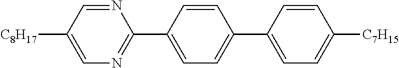 | 19.2 | 16.7 | 14.2 |
| 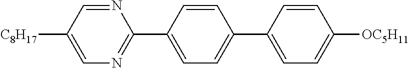 | 10.5 | 16.3 | 21.9 |
| 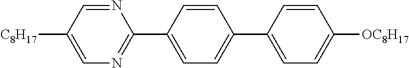 | 8.8 | 13.6 | 18.2 |

Mixtures BPPO-61-BPPO-63 shows following phase transitions:

| | |
|---|---|
| BPPO- 61 | 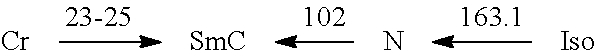 |
| BPPO- 62 | 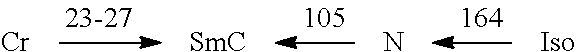 |
| BPPO- 63 | 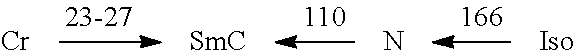 |

The inventors of the present disclosure have developed a new chiral compound with a formula:

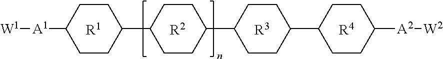

where,

n is 0 or 1;

R¹R², R³, and R⁴ each independently are 1,4-phenylene, optionally substituted with halogen or methyl at 2 or 3 positions, or pyrimidine-2,5-diyl, or pyridine-2,5-diyl, provided that at least one of the rings R¹ and R⁴ are different from unsubstituted 1,4-phenylene;

A¹ and A² are independently a single C—C bond, O, S, or an ester functional group;

W¹ and W² are chiral groups of shown absolute configuration selected either from the table below:

| W¹ | | W² |
|---|---|---|
| 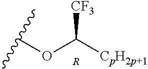 | 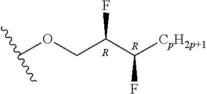 | 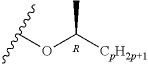 |
| 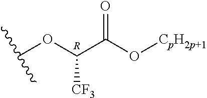 | 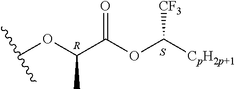 | 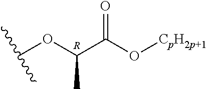 |
| 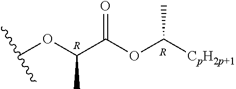 | 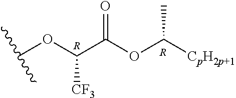 | |
| 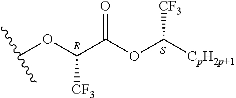 | 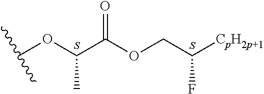 | |
| 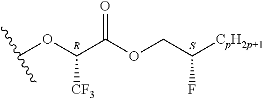 | 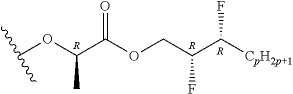 | |

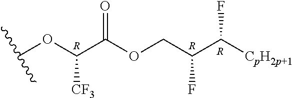

Or W¹ and W² are chiral groups with mirror configuration of each chiral center shown in the table below:

| W¹ | | W² |
|---|---|---|
| 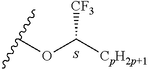 | 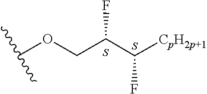 | 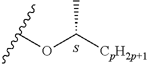 |
| 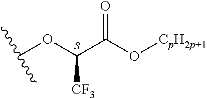 | 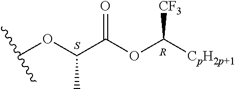 | 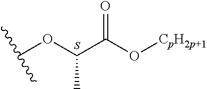 |
| 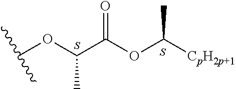 | 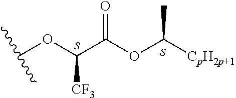 | |
| 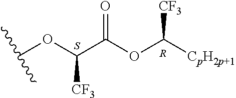 | 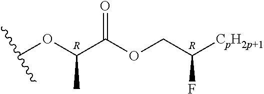 | |
| 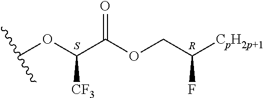 | 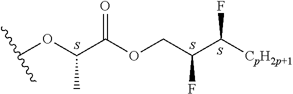 | |

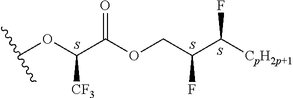

where CₚH₂ₚ₊₁ is alkyl group, p=2-14, and optionally where one or more CH₂ are independently replaced with CF₂, O, or —CO— groups provided that two O atoms are not linked together.

As an example of the chiral components that increase the tilt angle in FLC mixtures, a compound S-PDN-TFA-6 is of a formula:

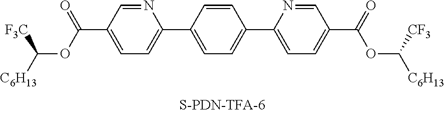

### Comparison of FLC Properties for Mixtures with Different Chiral Components

At the beginning, the invertors of the present disclosure studied all chiral compounds as a sole component of an FLC mixtures in order to optimized phase transitions, tilt angle and other important electrooptical properties. The comparison of the chiral components (S-DFT-TFA-6, with S-PDN-TFA-6) in their effect on FLC performance was carried out in the same host BPP-4 at a similar concentration. Temperature dependencies of helix pitch and HTP are given on FIG. 3A-FIG. 5B.

FIGS. 3A and 3B shows temperature dependencies of helix pitch (3A) and HTP (3B) for the chiral dopants S-DFT-TFA-6 (FLC9-087) and S-PDN-TFA-6 (FLC9-146).

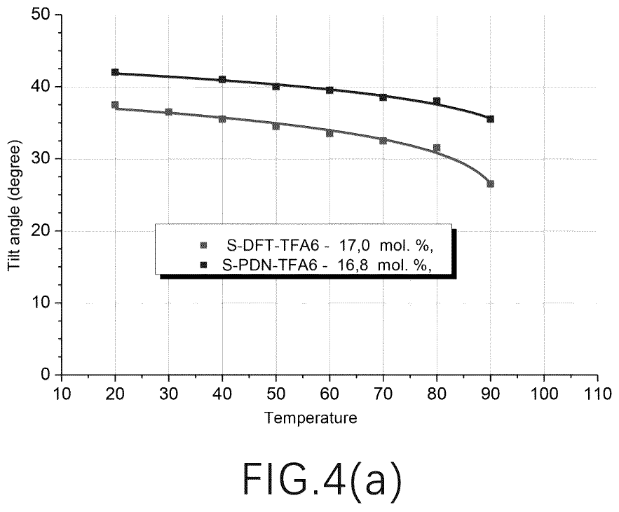

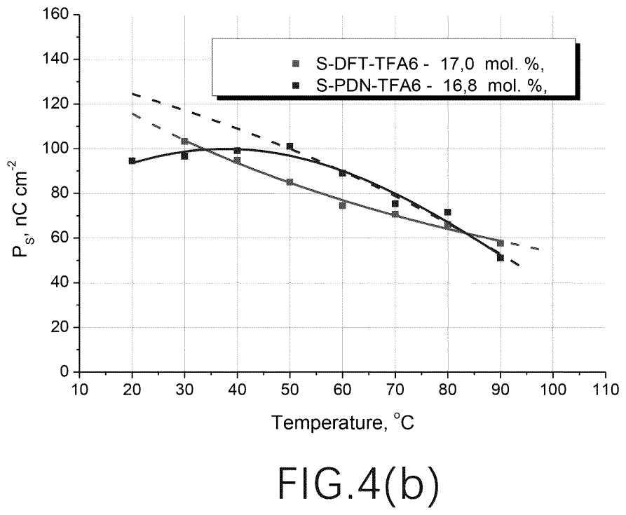

FLC properties of the mixtures are shown on FIGS. 4A and 4B.

FIGS. 4A and 4B shows temperature dependencies of tilt angle (4A) and spontaneous polarization (4B) for the FLC-9-087 containing the chiral dopant S-DFT-TFA-6 and FLC-9-146 with S-PDN-TFA-6 in BPP-4 host. At the plot (4B) the dotted line is approximation except 3 points at temperatures 20, 30 and 40° C.

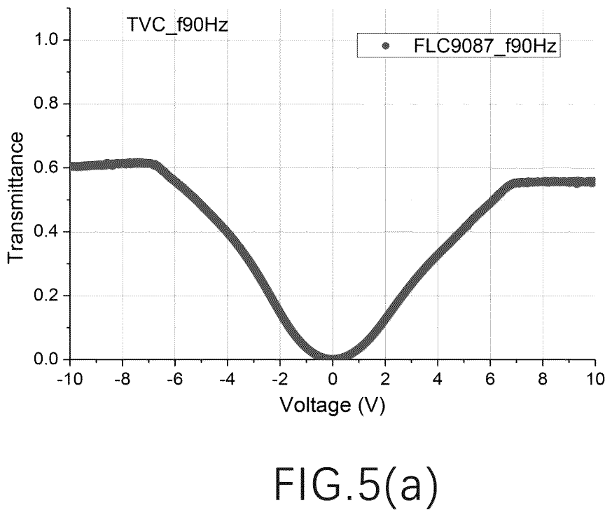

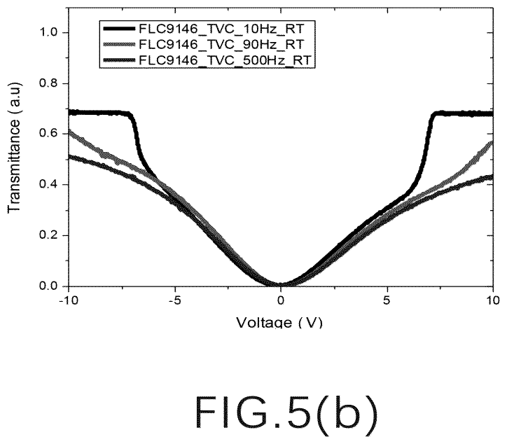

The comparison of electro-optical performance is given at FIGS. 5A and 5B.

FIGS. 5A and 5B shows dependencies of transmittance at 632 nm vs voltage of the FLC-9-087 (5A) and FLC-146 (5B).

Data collected at 25° C. are also given in Table 3.

TABLE 3
Electrooptical and ferroelectric characteristics
of the mixtures in BPP-4 of the dopants S-DFT-
TFA-6 (17 mol. %) and S-PDN-TFA-6 (16.8 mol. %)

| | | | | Unwinding | |
|---|---|---|---|---|---|
| Chiral dopant, | Tilt | On time | Off time | electric field, | Ps |
| Mixture code | (°) | (μs) | (μs) | Eᵤ, V/μm | (nC/cm²) |
| S-DFT-TFA-6, | 37.5 | 34 | 341 | 6.8 | 109.2 |
| 9-087 | | | | | |
| S-PDN-TFA-6, | 42 | 55 | 94 | 7.5 | 94.5 |
| 9-146 | | | | | |

Electric field strength, Eu = V/1.5, where 1.5 is cell gap in μm.

The comparative examination shows that new chiral dopant (S-PDN-TFA-6) meets expectations. Most of the parameters are either better or on the similar level as are with S-DFT-TFA-6. Especially should be noted tilt value (>40°) and its flatter temperature dependence than that for the S-DFT-TFA-6, see FIG. 4A. Few issues, as a non-linear Ps dependence (FIG. 4B) in S-PDN-TFA-6 containing mixture can be associated with induction of an additional helix ordering, like ferroelectric phases, which also follows from the bent on TVC curve at about 6 V, see FIG. 5B.

In order to precise control the helix twist and spontaneous polarization, the inventors of the present disclosure deployed FLC mixtures containing at least two chiral compounds with formulae:

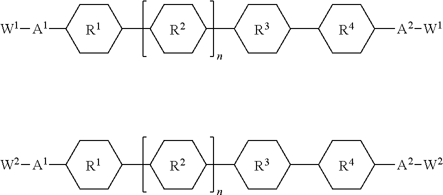

where

n is 0 or 1;

R¹, R², R³, and R⁴ each independently are 1,4-phenylene, optionally substituted with halogen or methyl at 2 or 3 positions, or pyrimidine-2,5-diyl, or pyridine-2,5-diyl, provided that at least one of the rings R¹ and R⁴ are different from unsubstituted 1,4-phenylene;

A¹ and A² are independently a single C—C bond, O, S, or an ester functional group;

W¹ and W² are chiral groups of shown absolute configuration selected either from the table below:

| W¹ | | | W² |
|---|---|---|---|
| 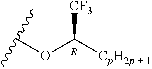 | 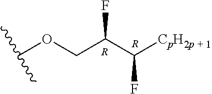 | 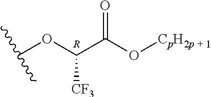 | 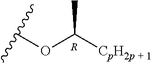 |
| 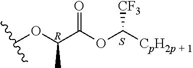 | 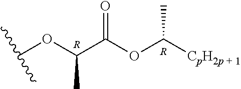 | 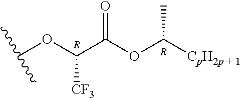 | 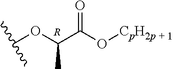 |
| 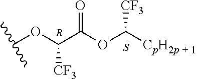 | 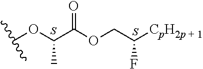 | 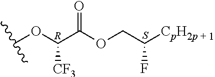 | |

| | |
|---|---|
| 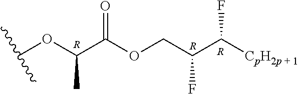 | 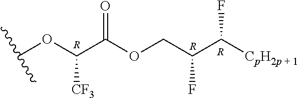 |

Or chiral groups with mirror configuration of each chiral centre shown in the table below:

| W¹ | | | W² |
|---|---|---|---|
| 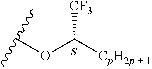 | 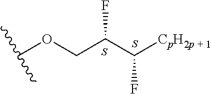 | 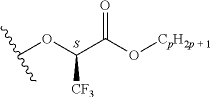 | 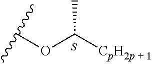 |
| 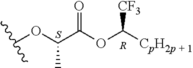 | 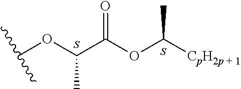 | 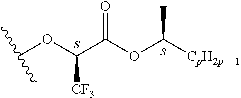 | 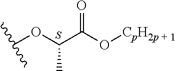 |
| 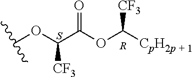 | 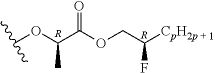 | 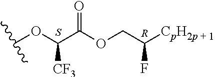 | |

| | |
|---|---|
| 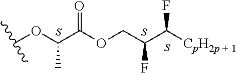 | 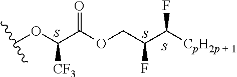 |

where CₚH₂ₚ₊₁ is alkyl group, p=2-14, and optionally where one or more CH₂ are independently replaced with CF₂, O, or —CO— groups provided that two O atoms are not linked together.

Comparison of FLC properties for mixtures based on different non-chiral components (Hosts)

Test of the new hosts (non-chiral mixtures of SmC LC compounds) has been carried out using S-DFT-TFA-6 at similar concentrations. As we mentioned above, the most promising candidate to expand SmC range are alkyloxy derivatives of BPP, particularly, BPPO-6#. The data are collected in Table 4, where results taken in BPP-4 host are given as a reference.

TABLE 4
Properties of the FLC mixtures based on alkyloxy-containing biphenyl
pyrimidine host (for host see Example 7)

| | | Content | | | | | | | | |
|---|---|---|---|---|---|---|---|---|---|---|
| | | of | | | | | | | On | |
| | | alkyloxy | | | Phase transition | | Helix | | time | Ps, |
| Mixture | | BPP, | Chiral | Conc., | temperatures | | pitch, | Tilt | (μ s)/ | nC/ |
| code | Host | mol % | dopant, | mol % | mp | T_{SmC*} | nm | (°) | V | cm² |
| 9-196 | BPP-4 | 0 | S-DFT- | 25 | 10.0 | 104 | 96 | 40 | 275 | 189 |
| | | | TFA-6 | | | | | | | |
| 11-127 | BPPO- | 19.3 | Fodta-6 | 25 | 9.3 | 106 | 108.7 | 37 | 310 | 165 |
| | 61 | | | | | | | | | |
| 11-110 | BPPO- | 19.3 | S-DFT- | 24.9 | 7.3 | 105 | 115 | 40.3 | 250 | 175 |
| | 61 | | TFA-6 | | | | | | | |
| 11-110- | BPPO- | 29.9 | S-DFT- | 24.8 | 12.0 | 116.6 | 118.3 | 40.5 | 300 | 188 |
| M2 | 62 | | TFA-6 | | | | | | | |
| 11-110- | BPPO- | 40.1 | S-DFT- | 25.3 | 16.4 | 115.8 | 115.6 | 42 | 310 | 179 |
| M3 | 63 | | TFA-6 | | | | | | | |
| 11-066 | BPP-4 | 0 | S-PDN- | 25.0 | 12.0 | 99.8 | 99 | 42.5 | 100 | 164 |
| | | | TFA6 | | | | | | | |
| 11-115 | BPPO- | 19.3 | S-PDN- | 24.8 | 13.6 | 101.9 | 124 | 41.5 | 130 | 154 |
| | 61 | | TFA6 | | | | | | | |

As it can be seen from Table 4, adding of alkyloxy-biphenyl pyrimidines into the host not only improve SmC transition point, but in combination with certain chiral components also increase a tilt angle, even at concentrations as low as 20-30%—compare mixture 9-196 with 11-110-M1-11-110-M3.

### FLC Mixtures Containing Several Chiral Dopants for the Kerr Effect

To precise control the helix twist and spontaneous polarization, we deployed two types

of chiral dopants that are of opposite sign of twisting and same sign of spontaneous polarization, see Table 5.

TABLE 4
Signs of helix twisting and spontaneous polarization
(P_{S}) for the deployed types of chiral dopants

| Chiral dopant | Sign of helix twisting | Sign of P_{S} |
|---|---|---|
| S-DFT-TFA-6 | + | + |
| S-PDN-TFA-6 | + | + |
| S-Lact-2 | − | + |
| S-Lact-4 | − | + |

The data for the FLC mixtures containing these two types of chiral dopants are given

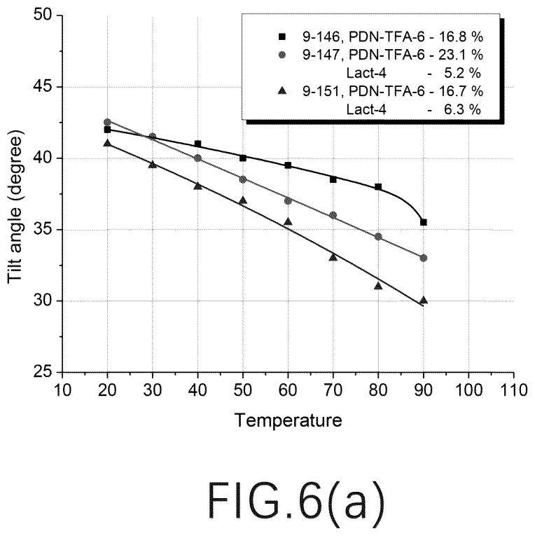

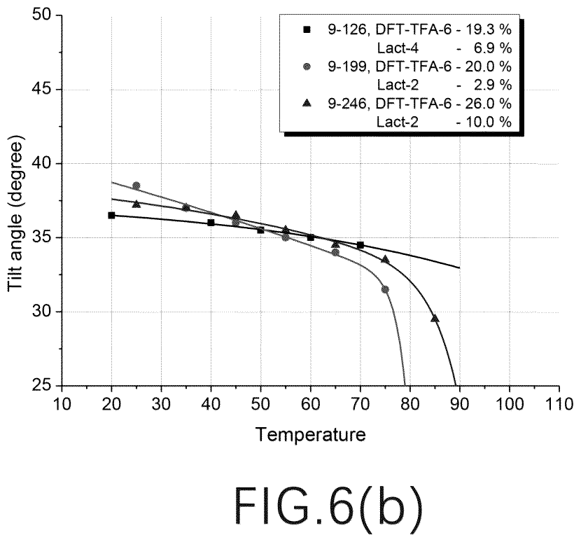

in Table 6 and FIGS. 6A-10C.

FIGS. 6A and 6B shows temperature dependencies of tilt angle for the FLC mixtures comprising binary chiral dopants: S-Lact-n with S-PDN-TFA-6 (6A) and S-Lact-n with S-DFT-TFA-6 (6B). Single-chiral dopant mixture (9-146) is given as a reference.

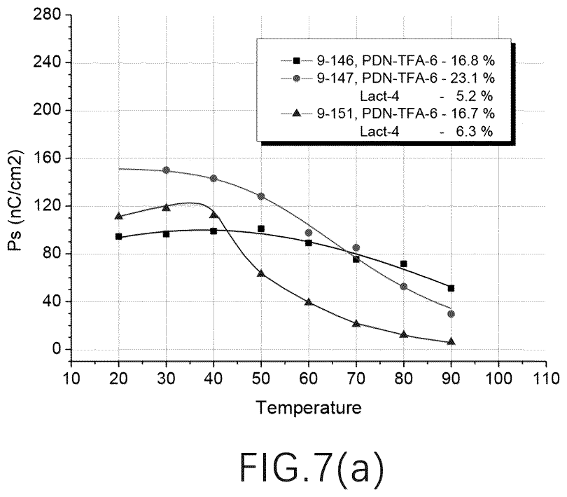

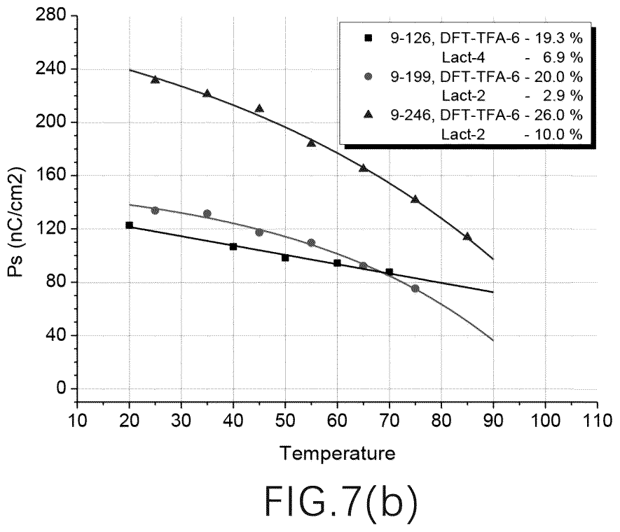

FIGS. 7A and 7B shows temperature dependencies of spontaneous polarization for the FLC mixtures comprising binary chiral dopants: S-Lact-n with S-PDN-TFA-6 (7A) and S-Lact-n with S-DFT-TFA-6 (7B). Single-chiral dopant mixture 9-146 is given as a reference.

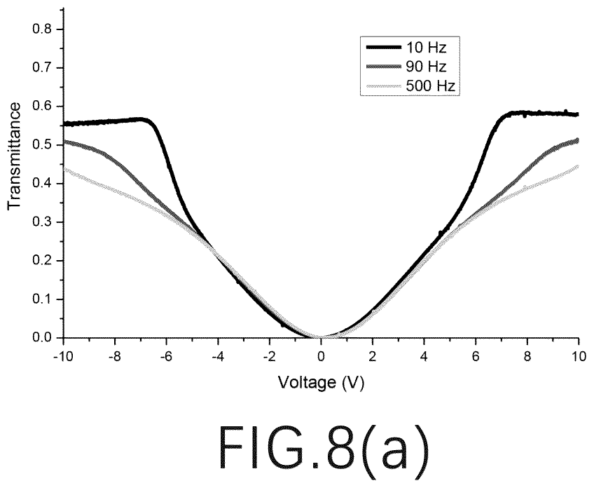

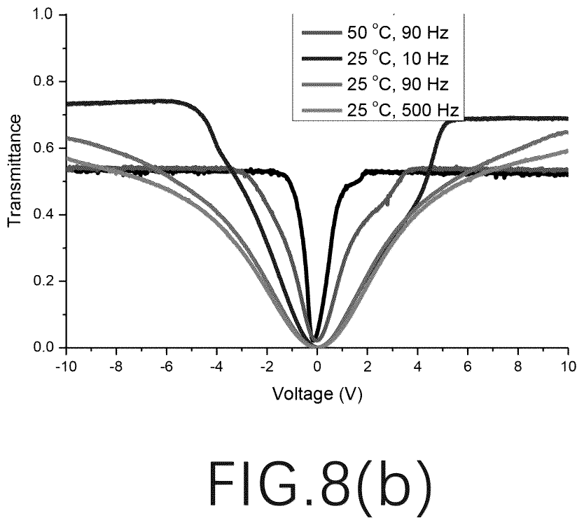

As it can be seen from FIG. 7A, the S-PDN-TFA-6 chiral dopant as individually, as in combination with S-Lact-4, also shows non-monotonic type of PS(T) dependencies. The transmittance voltage curves for these mixtures at room temperatures have also an inflection before saturation (see FIGS. 8A and 8B), which point out on possible induction of ferroelectric phases.

FIGS. 8A and 8B shows dependencies of transmittance at 632 nm vs voltage of mixtures comprising binary chiral dopants S-PDN-TFA-6/S-Lact-4 9-147 (8A) and 9-151 (8B), (for the rations values see Table 6).

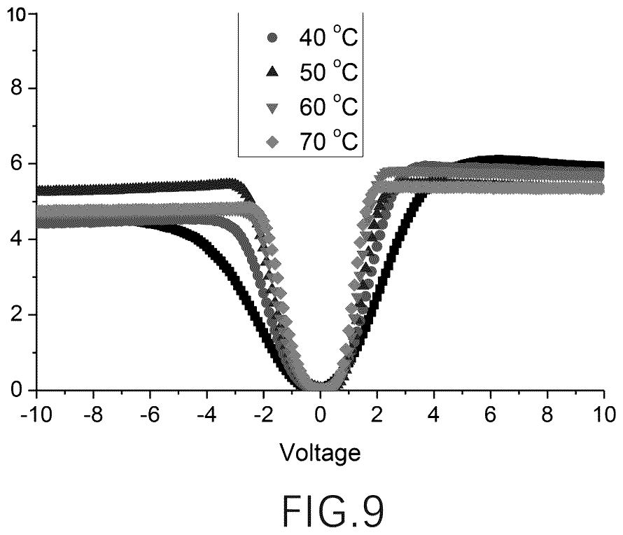

Conversely, the mixture with S-DFT-TFA-6/S-Lact-4 has not revealed any of such inflections, see FIG. 9. Thus, for further studying DFT type of chiral dopant was chosen as a main chiral component, although it shows lower tilt angle than dopant of PDN type.

FIGS. 9 shows dependencies of transmittance at 632 nm vs voltage of mixture comprising binary chiral dopants S-DFT-TFA-6/S-Lact-4 (9-126) at different temperatures.

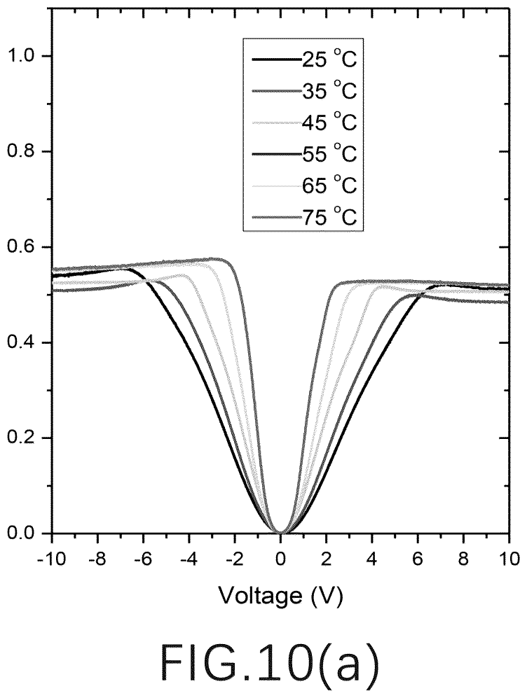

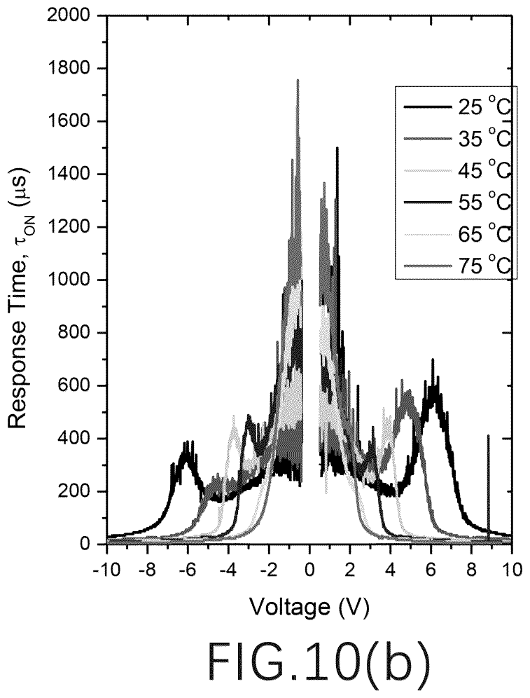

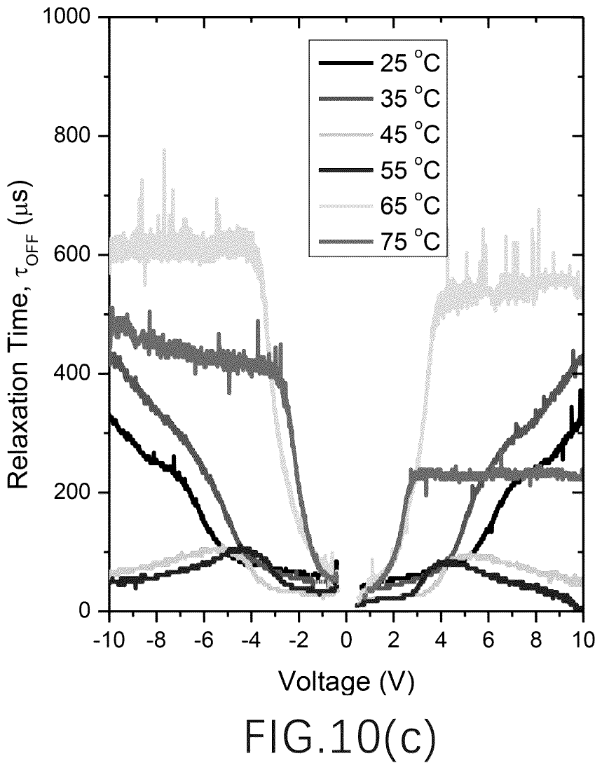

FIGS. 10A, 10B and 10C show evolution of electrooptical characteristics with temperature for the FLC-9-199 mixture measured at 90 HZ, including transmittance vs Voltage (10A), Response time_on vs Voltage (10B), and Response time_off vs Voltage (10C).

As we have noted in our previous report, the S-Lact-4 has rather low polarization power, thus it brings a weak impact to the total P_{S} in the mixtures. In an attempt to improve the low impact of Lactate type dopants, we have tried Lactate with a shorter terminal tail as the second component, namely S-Lact-2, see Table 6.

Indeed, S-Lact-2 shows better performance in the mixtures with S-DFT-TFA-6 dopant than that for S-Lact-4. Surprisingly, S-Lact-2 behaves in the mixtures, like it has the HTP=−45 μm⁻¹ and Polarization power of 6.5 nC/(cm² mol %) that are comparable with those values of 5-DFT-TFA-6 dopants, although individual |HTP| of S-Lact-2 does not exceed 12-15 μm⁻¹.

TABLE 5
Properties of the FLC mixtures comprising several chiral dopants

| | | | | | | | | | | K_{Kerr}, |
|---|---|---|---|---|---|---|---|---|---|---|
| | | | | Phase | | | | | | nm/ |
| | | | | transition | | Helix | | On | | V² |
| Mixture | | Chiral | Conc., | temperatures | | pitch, | Tilt | time | Ps, nC/ | at 1550 |
| code | Host | dopant, | mol % | mp | T_{SmC*} | nm | (°) | (μs) | cm² | nm |
| 9-112 | BPP-6 | S-DFT- | 15.0 | 12.3 | 99.5 | 270 | 38.5 | 130 | 127.6 | 51 |
| | | TFA-6 | | | | | | | | |
| | | S-Lact-4 | 11.2 | | | | | | | |
| 9-126 | BPP-6 | S-DFT- | 19.3 | 9.7 | 99 | 139 | 36.5 | 51 | 122.7 | 39 |
| | | TFA-6 | | | | | | | | |
| | | S-Lact-4 | 6.9 | | | | | | | |
| 9-147 | BPP-6 | S-PDN- | 23.1 | 10.7 | 101.5 | 121 | 42.5 | 136 | 150 | 67 |
| | | TFA-6 | | | | | | | | |
| | | S-Lact-4 | 5.2 | | | | | | | |
| 9-151 | BPP-6 | S-PDN- | 16.7 | 12 | 99.7 | 155 | 41 | 51 | 115 | 61 |
| | | TFA-6 | | | | | | | | |
| | | S-Lact-4 | 6.3 | | | | | | | |
| 9-199 | BPP-4 | S-DFT- | 20 | 10.8 | 103.2 | 141 | 38 | ~190 | 129.9 | 87 |
| | | TFA-6 | | | | | | | | |
| | | S-Lact-2 | 2.9 | | | | | | | |
| 9-246 | BPP-4 | S-DFT- | 26 | 4.2 | ~94 | 155 | 37.2 | 150 | 231 | 240 |
| | | TFA-6 | | | | | | | | |
| | | S-Lact-2 | 10 | | | | | | | |
| 11-009 | BPP-4 | S-PDN- | 24.0 | 8.4 | 80 | 164.5 | 40.5 | 55 | 237.4 | 280 |
| | | TFA-6 | | | | | | | | |
| | | S-Lact-2 | 11.1 | | | | | | | |
| 11-015 | BPP-4 | S-DFT- | 7.8 | 4.5 | 83 | 122 | 43 | 45 | 184 | 235 |
| | | TFA-6 | | | | | | | | |
| | | S-PDN- | 23.9 | | | | | | | |
| | | TFA-6 | | | | | | | | |
| | | S-Lact-2 | 10.5 | | | | | | | |

As we have assumed, further Kerr constant improvement over 51 nm/V² can be achieved by increasing P_{S} value in FLC mixtures over 200 nC/cm² keeping helix pitch value in the range of 150-200 nm, for the results see Table 6 and FIG. 5A-FIG. 6B. Among these results, outstanding performance was revealed for the mixtures 11-009 and 11-015, where the high Kerr constant are accompanied by tilt values over 40 degrees.

In summary, we have developed approaches for design of Kerr effect FLC materials that are promising in application as a phase modulator in telecommunication devices.

Following are detailed examples of the chiral components, non-chiral host and FLC mixtures.

### Example 1 Synthesis of bis((S)-1-butoxy-1-oxopropan-2-yl) [1,1′:4′,1″-terphenyl]-4,4″-dicarboxylate (S-Lact-4)

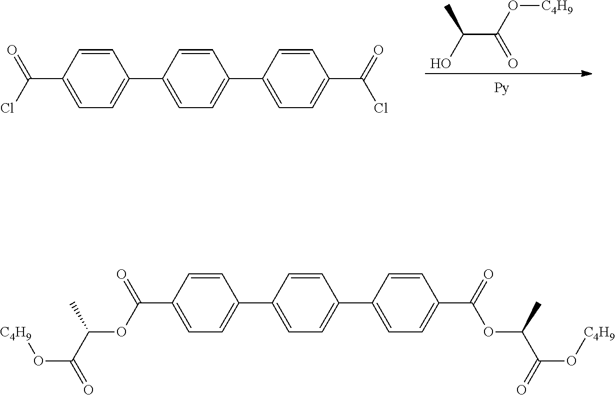

A mixture of 4.05 g (11.4 mmol) of 4,4″-terphenyldicarboxylic acid dichloride (obtained as it was described by Cambell, T. W. (1960). J. Am. Chem. Soc., 82, 3126.) and 6.67 g (45.6 mmol) of S-butyl lactate (Acros) in 60 mL of dry toluene is heated to reflux and then slowly added a solution of 18 mL of pyridine in 25 mL of toluene. After completion of adding, the mixture is refluxed for 6 h, cooled down to an ambient temperature, evaporated to dryness and residual material is purified by flash-chromatography on silica gel using dichloromethane as an eluent. The solution in dichloromethane is evaporated to dryness and recrystallized two times from 80 ml of acetonitrile. Obtained crystalline material is dissolved in toluene and filtered through 0.2 μm PTFE filter to remove dust particles, evaporate to dryness and dry in vacuum desiccator at 0.5-1 mbar for 12 h. Yield of colorless solid material is 4.06 g (62%).

Example 2 Synthesis of bis((S)-1-ethyloxy-1-oxopropan-2-yl) [1,1′:4′,1″-terphenyl]-4,4″-dicarboxylate (S-Lact-2) is carried out essentially following to the protocol described in Example 1 with the difference that S-ethyllactate is used instead of S-butyllactate.

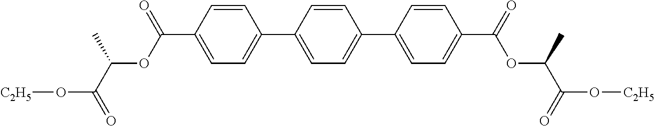 [SMILES: CCOC(=O)[C@H](C)OC(=O)c1ccc(-c2ccc(-c3ccc(C(=O)O[C@@H](C)C(=O)OCC)cc3)cc2)cc1]

Quantities: 4,4″-terphenyldicarboxylic acid dichloride 3.55 g (10 mmol); S-ethyllactate 3.55 g (30.0 mmol); pyridine 15 mL; Yield of the product is 3.53 g (68%).

### Example 2. Synthesis of bis-(S-2-trifluoromethyl-heptyl) 3,3″-difluoro-[1,1′:4′,1″-terphenyl]-4,4″-dicarboxylate (S-DFT-TFA-6) was Carried Out Accordingly To The Scheme Below

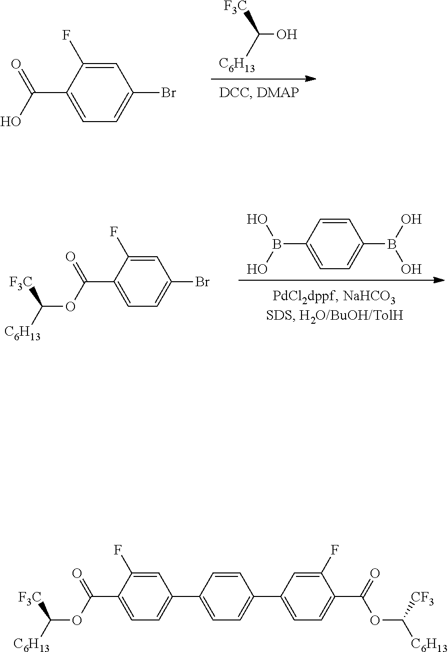

S-2-(trifluoromethyl)heptyl-2-fluoro-4-bromobenzoate. A solution of 3.27 g (15.8 mmol) of dycyclohexyl-carbodiimide (DCC) in 20 ml of dry dichloromethane (DCM) is added dropwise to a stirred and cooled (ice-water) mixture of 2.89 g (13.2 mmol) of 2-fluoro-4-bromobenzoic acid, 2.28 g (12.4 mmol) of S-2-(trifluoromethyl)-heptanol and 5 mg of 4-N,N-dimethylaminopyridine in 30 ml of DCM. After finishing addition, the mixture is stirred while reaction is completed (monitoring by TLC), then filtered through the short plug of silica gel. The silica gel washed additionally with 150 ml of DCM, the combined solutions in DCM is evaporated to dryness furnishing 5.2 g of oil, which is solidified upon storage. The ester was used on the next step without additional purification.

Bis-(S-2-trifluoromethyl-heptyl) 3,3″-difluoro-[1,1′:4′,1″-terphenyl]-4,4″-dicarboxylate. Mixture of 2.22 g (5.8 mmol) of S-2-(trifluoromethyl)heptyl-2-fluoro-4-bromobenzoate, 0.4 g (2.4 mmol) of 1,4-phenylenediboronic acid, 300 mg of sodium dodecylsulphate (SDS), 171 mg of PdCl₂dppf, 5 ml of 1-butanol, 10 ml of water, and 30 ml of toluene is thoroughly degassed (3 cycles of pumping out to — 100 mbar and filling with N₂), then heated to reflux and added dropwise a degassed solution of 2.9 g (34.8 mmol) of NaHCO₃ in 20 ml of water. Reaction mixture is refluxed additionally for 2 h, then cooled down to ambient temperature and separated organic layer. The water layer is extracted 3 times with toluene. Combined organic extracts is washed with water, dried over Na₂SO₄, filtered through short plug of silica gel, washed silica gel with toluene and evaporated toluene filtrate to dryness. The residual yellowish oil is purified by column chromatography on silica gel (50×2 cm, eluent Toluene:Hexane 1:1 w/w), furnished 1.00 g (62%) of the product as a colourless oil.

Example 4 Syntheis of bis((S)-1,1,1-trifluorooctan-2-yl) 6,6′-(1,4-phenylene)dipicolinate (S-PDN-TFA-6) is carried out essentially following to the protocol described in Example 3 with the difference that 6-bromonicotinic acid is used instead of 2-fluoro-4-bromobenzoic acid.

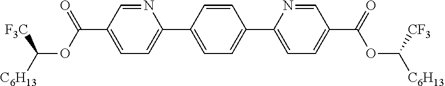

Quantaties: 6-bromonicotinic acid 2.024 g (10 mmol); S-2-(trifluoromethyl)heptanol 1.76 g (9.54 mmol); 1,4-phenylene diboronic acid 0.624 g (3.76 mmol). Yield of the product is 1.0 g (40.7%), mp. 57° C.

### Example 3 Mixture of 6 biphenylpyrimidines BPP-6, Used as a Non-Chiral Host

| | Content of the component, |
|---|---|
| Chemical formulae | mol.% |
| 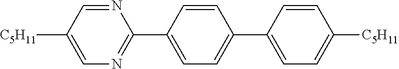 | 4.0 |
| 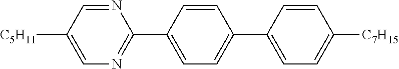 | 5.0 |
| 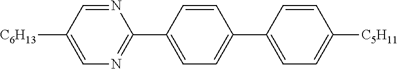 | 49.5 |
| 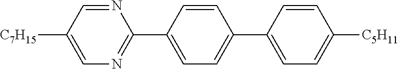 | 19.8 |
| 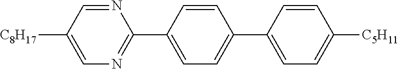 | 2.0 |
| 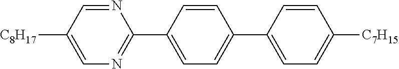 | 19.8 |

Mixture BPP-6 shows the following phase transitions:

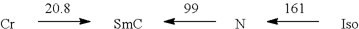

### Example 4 Mixture of 4 biphenylpyrimidines BPP-4, Used as a Non-Chiral Host

| | Content of the component, |
|---|---|
| Chemical formulae | mol.% |
| 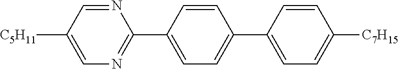 | 17.7 |
| 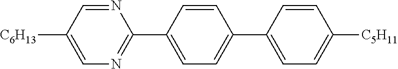 | 40.1 |
| 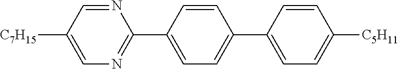 | 18.2 |
| 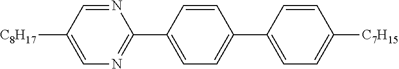 | 24 |

Mixture BPP-4 shows the following phase transitions:

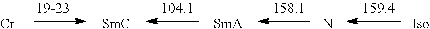

### Example 5 The Mixture of 6 Three-Ring Biphenylpyrimidines Including to O-alkyl Substituted Compounds is Shown Below (Mixture BPP-60):

| | Content of the component, mol.% | | |
|---|---|---|---|
| | BPPO- | BPPO- | BPPO- |
| Chemical formulae | 61 | 62 | 63 |
| 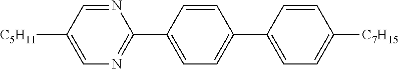 | 14.4 | 12.5 | 10.7 |
| 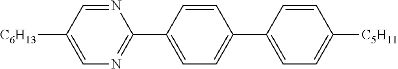 | 32.5 | 28.3 | 24.2 |
| 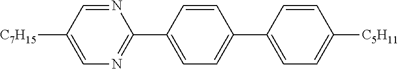 | 14.6 | 12.6 | 10.8 |
| 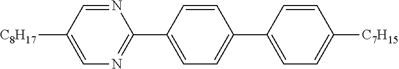 | 19.2 | 16.7 | 14.2 |
| 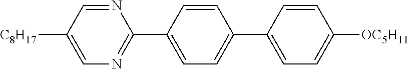 | 10.5 | 16.3 | 21.9 |
| 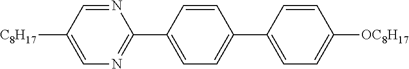 | 8.8 | 13.6 | 18.2 |

Mixtures shows the following phase transitions:

| | |
|---|---|
| BPPO-61 | 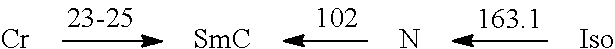 |
| BPPO-62 | 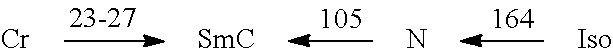 |
| BPPO-63 | 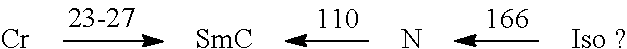 |

### Example 6 FLC Mixture FLC-9-087 Containing One Chiral Guest

The FLC mixture has following compositions:

| | Content of the component, |
|---|---|
| Chemical formulae | mol.% |
| BPP-6 | 83 |
| 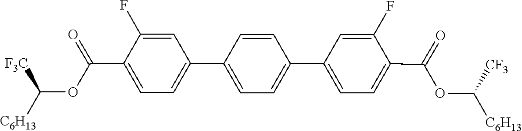 | 17 |

Mixture FLC-9-087 shows following properties (at 25° C.):

| | |
|---|---|
| Phase transitions | 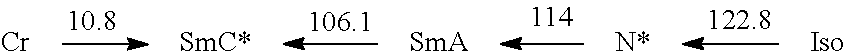 |
| Helix pitch, p0, nm | 134 |
| Tilt angle, θ, degree | 37.5 |
| Critical voltage of | 6.8 |
| helix unwinding, Vc, | |
| V | |
| Spontaneous | 109.2 |
| polarization, P_{S}, | |
| nC/cm² | |
| Switching on time, | 34 |
| τ_{ON}, μs | |

### Example 7 FLC Mixture FLC-9-091, Containing Two Chiral Guests

The FLC mixture has following composition.

| | Content of the component, |
|---|---|
| Chemical formulae | mol.% |
| BPP-6 | 77.4 |
| 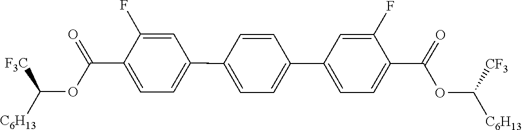 | 17.0 |
| 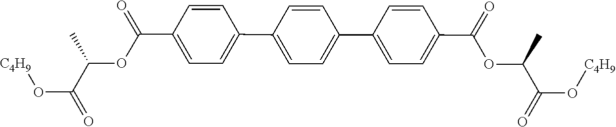 | 5.6 |

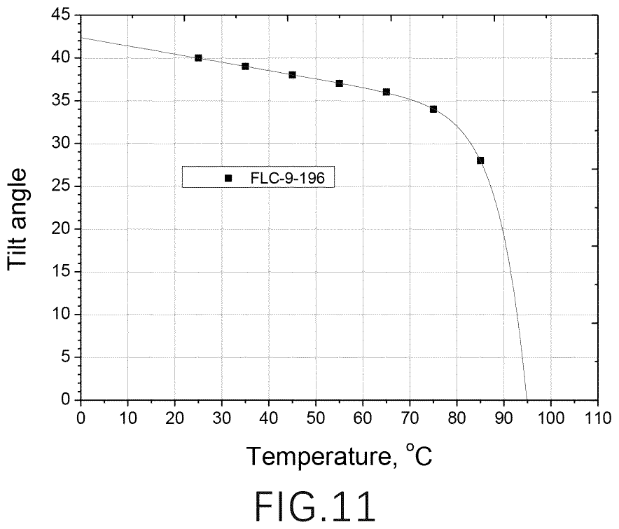

Mixture FLC-9-091 shows following properties (at 25° C.), FIG. 11 shows tilt temperature dependence for the mixture FLC-9-091, see Example 9.

| | |
|---|---|
| Phase transitions | 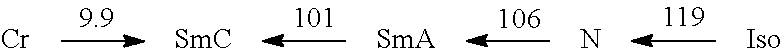 |
| Helix pitch, p0, nm | 172 |
| Tilt angle, θ, degree | 36.5 |
| Critical voltage of helix | 5.7 |
| unwinding, Vc, V | |
| Spontaneous | 110 |
| polarization, P_{S}, nC/cm² | |
| Switching on time, τ_{ON}, | 56 |
| μs | |
| Switching off time, τ_{OFF}, | 83 |
| μs | |

### Example 8 FLC Mixture FLC-9-112 Containing Two Chiral Guests

The FLC mixture has following composition.

| | Content of the component, |
|---|---|
| Chemical formulae | mol.% |
| BPP-6 | 73.8 |
| 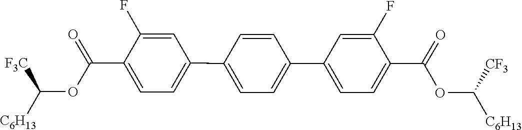 | 15 |
| 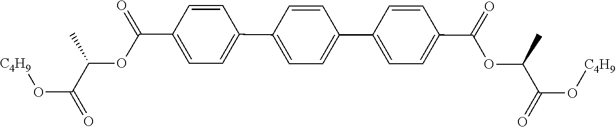 | 11.2 |

Mixture FLC-9-112 shows following properties (at 25° C.):

| | |
|---|---|
| Phase transitions | 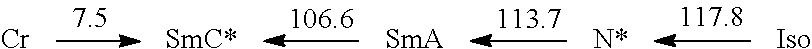 |
| Helix pitch, p0, nm | 270 |
| Tilt angle, θ, degree | 38.5 |
| Critical voltage of | 5.7 |
| helix unwinding, Vc, | |
| V | |
| Spontaneous | 127.6 |
| polarization, P_{S}, | |
| nC/cm² | |
| Switching on time, | 130 |
| τ_{ON}, μs | |
| Switching off time, | 86 |
| τ_{OFF}, μs | |

### Example 9 FLC Mixture FLC-9-126 Containing Two Chiral Guests

The FLC mixture has following composition.

| | Content of the component, |
|---|---|
| Chemical formulae | mol.% |
| BPP-6 | 73.8 |
| 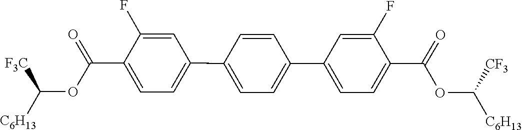 | 19.3 |
| 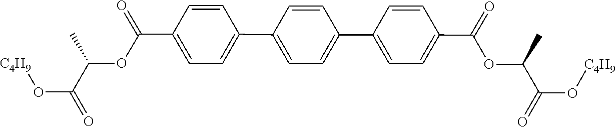 | 6.9 |

Mixture FLC-9-126 shows following properties (at 25° C.):

| | |
|---|---|
| Phase transitions | 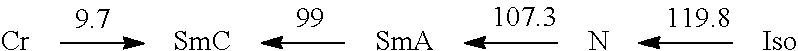 |
| Helix pitch, p0, nm | 139 |
| Tilt angle, θ, degree | 36.5 |
| Critical voltage of helix | 4.6 |
| unwinding, Vc, V | |
| Spontaneous | 122.7 |
| polarization, P_{S}, nC/cm² | |
| Switching on time, τ_{ON}, | 34 |
| μs | |
| Switching off time, τ_{OFF}, | 661 |
| μs | |

### Example 10 FLC Mixture FLC-9-127 Containing Two Chiral Guests

The FLC mixture has following composition.

| | Content of the component, |
|---|---|
| Chemical formulae | mol.% |
| BPP-6 | 74.8 |
| 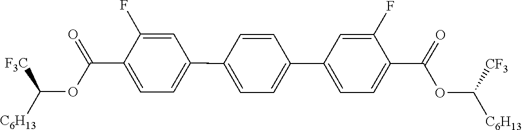 | 19.7 |
| 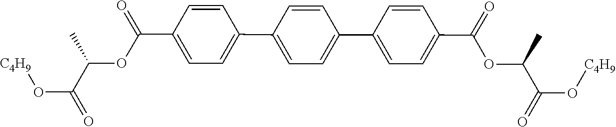 | 5.5 |

Mixture FLC-9-127 shows following properties (at 25° C.):

| Phase transitions | 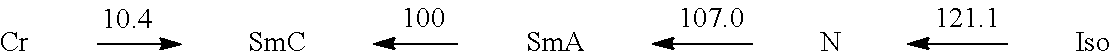 |
|---|---|
| Helix pitch, p0, nm | 134 |
| Tilt angle, θ, degree | 37 |
| Critical voltage of helix | 5.4 |
| unwinding, Vc, V | |
| Spontaneous polarization, | 117.5 |
| Ps, nC/cm² | |
| Switching on time, τ_{ON}, μs | 310 |

### Example 11 FLC Mixture FLC-9-146 Based on Six-Component Host

The mixture has following composition.

| | Content of the |
|---|---|
| | component, |
| Chemical formulae | mol. % |
| BPP-6 | 83.2 |
| 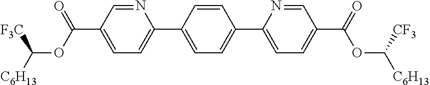 | 16.8 |

Mixture FLC-9-146 shows following properties (at 25° C.):

| Phase transitions | 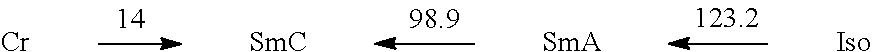 |
|---|---|
| Helix pitch, p0, nm | 123 |
| Tilt angle, θ, degree | 42.0 |
| Critical voltage of | 7.5 |
| helix unwinding, | |
| Vc, V | |
| Spontaneous | 94.5 |
| polarization, | |
| Ps, nC/cm² | |

### Example 12 FLC Mixture FLC-9-147 Containing Two Chiral Guests

The FLC mixture has following composition.

| | Content of the |
|---|---|
| | component, |
| Chemical formulae | mol. % |
| BPP-6 | 71.7 |
| 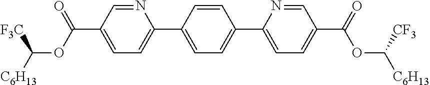 | 23.1 |
| 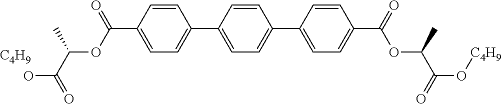 | 5.2 |

Mixture FLC-9-147 shows following properties (at 25° C.):

| Phase transitions | 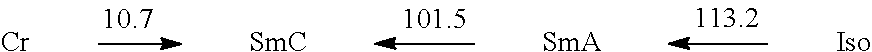 |
|---|---|
| Helix pitch, p0, nm | 121 |
| Tilt angle, θ, degree | 42.5 |
| Critical voltage of | 6.8 |
| helix unwinding, | |
| Vc, V | |
| Spontaneous | 150 |
| polarization, | |
| Ps, nC/cm² | |
| Switching on time, | 136 |
| τ_{ON}, μs | |

### Example 13 FLC Mixture FLC-9-151 Containing Two Chiral Guests

The FLC mixture has following composition.

| | Content of the |
|---|---|
| | component, |
| Chemical formulae | mol. % |
| BPP-6 | 73.8 |
| 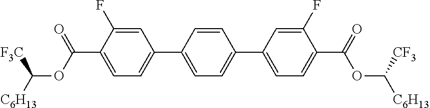 | 16.7 |
| 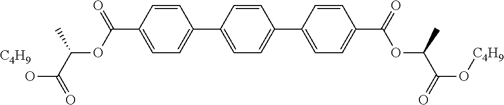 | 6.3 |

Mixture FLC-9-151 shows following properties (at 25° C.):

| Phase transitions | 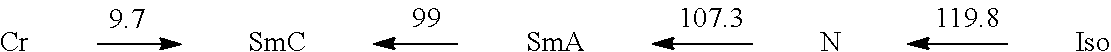 |
|---|---|
| Helix pitch, p0, nm | 155 |
| Tilt angle, θ, degree | 41 |
| Critical voltage of helix | 5.4 |
| unwinding, Vc, V | |
| Spontaneous polarization, | 115 |
| Ps, nC/cm² | |
| Switching on time, τ_{ON}, μs | 51 |
| Switching off time, τ_{OFF}, μs | 202 |

### Example 14 FLC Mixture FLC-9-196 Based on Four-Component Host With One Chiral Guest.

FLC-9-196 was used as a reference mixture.

The FLC mixture has following composition.

| | Content of the |
|---|---|
| | component, |
| Chemical formulae | mol. % |
| BPP-4 | 75 |
| 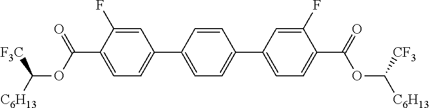 | 25 |

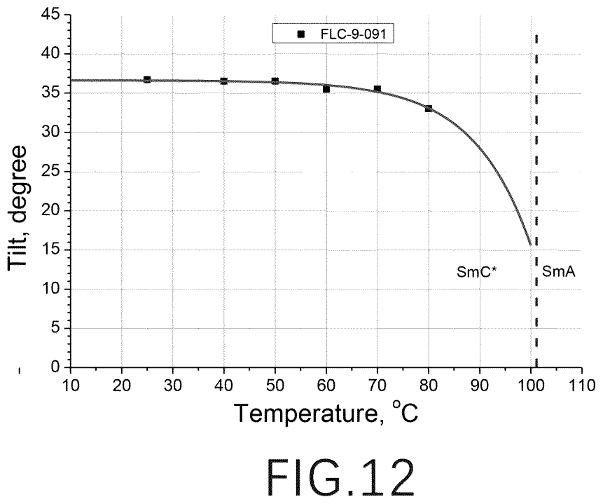

Mixture FLC-9-196 shows following properties (at 25° C.). FIG. 12 shows a tilt temperature dependence for the mixture FLC-9-196.

| Phase transitions | 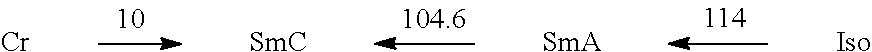 |
|---|---|
| Helix pitch, p0, nm | 95 |
| Tilt angle, θ, degree | 40 |
| Critical voltage of | 6.8 |
| helix unwinding, | |
| Vc, V | |
| Spontaneous | 131.6 |
| polarization, | |
| Ps, nC/cm² | |
| Switching on time, | 275 |
| τ_{ON}, μs | |
| Switching off time, | 62 |
| τ_{OFF}, μs | |

### Example 15 FLC Mixture FLC-9-199 Containing Two Chiral Guests

The FLC mixture has following composition.

| | Content of the |
|---|---|
| | component, |
| Chemical formulae | mol. % |
| BPP-4 | 77.1 |
| 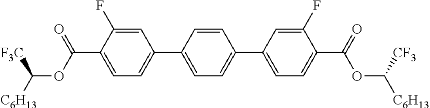 | 20 |
| 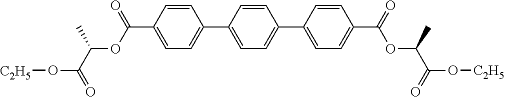 [SMILES: CCOC(=O)[C@H](C)OC(=O)c1ccc(-c2ccc(-c3ccc(C(=O)O[C@@H](C)C(=O)OCC)cc3)cc2)cc1] | 2.9 |

Mixture FLC-9-199 shows following properties (at 25° C.):

| Phase transitions | 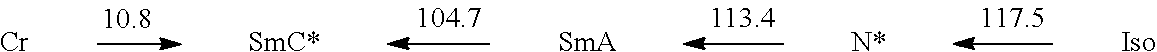 |
|---|---|
| Helix pitch, p0, nm | 141 |
| Tilt angle, θ, degree | 38 |
| Critical voltage of helix | 6.1 |
| unwinding, Vc, V | |
| Spontaneous polarization, | 129.9 |
| Ps, nC/cm² | |
| Switching on time, τ_{ON}, μs | 190 |
| Switching off time, τ_{OFF}, μs | 85 |

### Example 16 FLC Mixture FLC-9-246 Containing Two cChiral Guest

The FLC mixture has following composition.

| | Content of the |
|---|---|
| | component, |
| Chemical formulae | mol. % |
| BPP-4 | 77.1 |
| 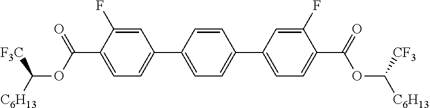 | 20 |
| 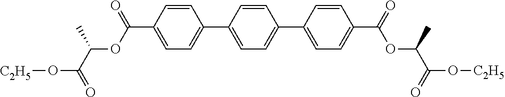 [SMILES: CCOC(=O)[C@H](C)OC(=O)c1ccc(-c2ccc(-c3ccc(C(=O)O[C@@H](C)C(=O)OCC)cc3)cc2)cc1] | 2.9 |

Mixture FLC- FLC-9-246 shows following properties (at 25° C.):

| Phase transitions | 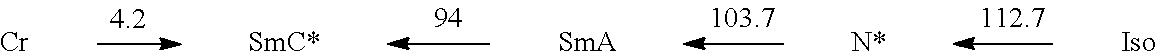 |
|---|---|
| Helix pitch, p0, nm | 155 |
| Tilt angle, θ, degree | 37.2 |
| Spontaneous polarization, | 231 |
| Ps, nC/cm² | |
| Switching on time, τ_{ON}, μs | 150 |
| Switching off time, τ_{OFF}, μs | 130 |

### Example 17 FLC Mixture FLC-11-009 Containing Two Chiral Guests

The FLC mixture has following composition.

| | Content of the |
|---|---|
| | component, |
| Chemical formulae | mol. % |
| BPP-4 | 63.8 |
| 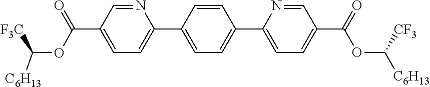 | 24.0 |
| 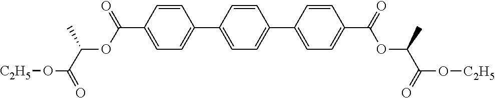 [SMILES: CCOC(=O)[C@H](C)OC(=O)c1ccc(-c2ccc(-c3ccc(C(=O)O[C@@H](C)C(=O)OCC)cc3)cc2)cc1] | 11.1 |

Mixture FLC-11-009 shows following properties (at 25° C.):

| Phase transitions | 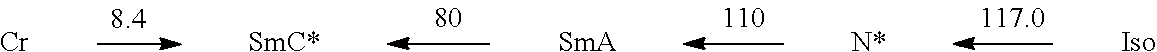 |
|---|---|
| Helix pitch, p0, nm | 164.5 |
| Tilt angle, θ, degree | 40.5 |
| Critical voltage of helix | 4.6 |
| unwinding, Vc, V | |
| Spontaneous polarization, | 237.4 |
| Ps, nC/cm² | |
| Switching on time, τ_{ON}, μs | 55 |
| Switching off time, τ_{OFF}, μs | 410 |

### Example 18 FLC Mixture FLC-11-015 Containing Three Chiral Guests

The FLC mixture has following composition.

| | Content of the component, |
|---|---|
| Chemical formulae | mol. % |
| BPP-4 | 57.8 |
| 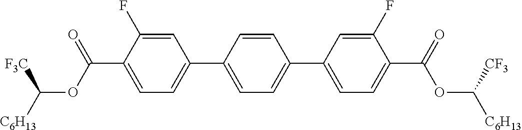 | 7.8 |
| 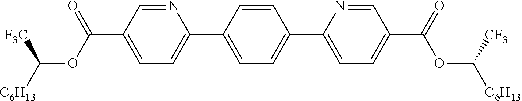 | 23.9 |
| 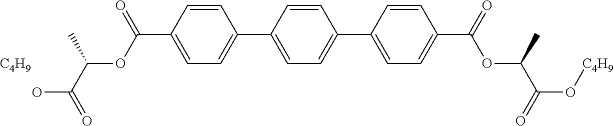 | 10.5 |

Mixture FLC-11-015 shows following properties (at 25° C.):

| | |
|---|---|
| Phase transitions | 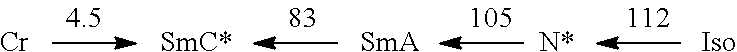 |
| Helix pitch, p0, nm | 122 |
| Tilt angle, θ, degree | 43 |
| Critical voltage of | 5.5 |
| helix unwinding, Vc, | |
| V | |
| Spontaneous | 184 |
| polarization, Ps, | |
| nC/cm² | |
| Switching on time, | 45 |
| τ_{ON}, μs | |

### Example 19 FLC Mixture FLC-11-066 Containing One Chiral Guest

The reference mixture.

The FLC mixture has following composition.

| | Content of the component, |
|---|---|
| Chemical formulae | mol. % |
| BPP-4 | 75 |
| 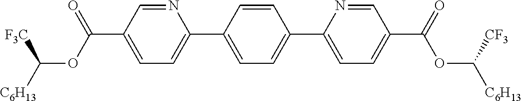 | 25 |

Mixture FLC-11-066 shows following properties (at 25° C.):

| | |
|---|---|
| Phase transitions | 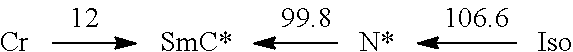 |
| Helix pitch, p0, nm | 99 |
| Tilt angle, θ, degree | 42.5 |
| Critical voltage of helix | 4.6 |
| unwinding, Vc, V | |
| Spontaneous | 164 |
| polarization, Ps, | |
| nC/cm² | |
| Switching on time, | 70 |
| τ_{ON}, μs | |

### Example 20 FLC Mixture FLC-11-110 Containing One Chiral Guest

The FLC mixture has following composition.

| | Content of the component, |
|---|---|
| Chemical formulae | mol. % |
| BPPO-61 | 75.1 |
| 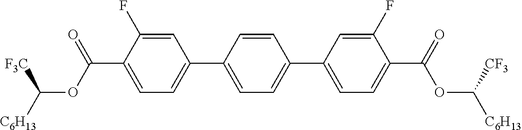 | 24.9 |

Mixture FLC-11-110 shows following properties (at 25° C.):

| | |
|---|---|
| Phase transitions | 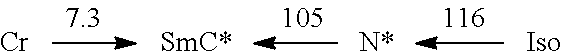 |
| Helix pitch, p0, nm | 115 |
| Tilt angle, θ, degree | 40.5 |
| Critical voltage of helix | 9 |
| unwinding, Vc, V | |
| Spontaneous | 175 |
| polarization, Ps, | |
| nC/cm² | |
| Switching on time, | 70 |
| τ_{ON}, μs | |

### Example 21 FLC Mixture FLC-11-110-M2 Containing One Chiral Guest

The FLC mixture has following composition.

| | Content of the component, |
|---|---|
| Chemical formulae | mol. % |
| BPPO-62 | 75.2 |
| 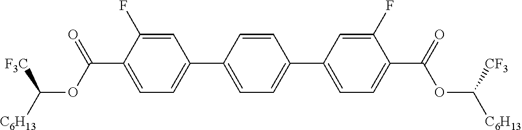 | 24.8 |

Mixture FLC-11-110-M2 shows following properties (at 25° C.):

| | |
|---|---|
| Phase transitions | 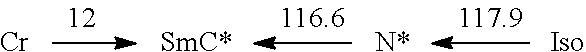 |
| Helix pitch, p0, nm | 115 |
| Tilt angle, θ, degree | 40.5 |
| Critical voltage of helix | 9 |
| unwinding, Vc, V | |
| Spontaneous | 175 |
| polarization, Ps, | |
| nC/cm² | |
| Switching on time, | 70 |
| τ_{ON}, μs | |

### Example 22 FLC Mixture FLC-11-110-M3 Containing One Chiral Guest

The FLC mixture has following composition.

| | Content of the component, |
|---|---|
| Chemical formulae | mol. % |
| BPPO-63 | 74.7 |
| 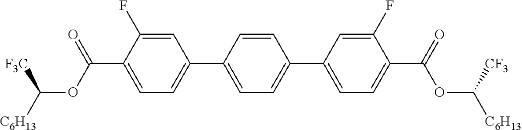 | 25.3 |

Mixture FLC-11-110-M3 shows following properties (at 25° C.):

| | |
|---|---|
| Phase transitions | 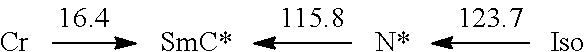 |
| Helix pitch, p0, nm | 115.6 |
| Tilt angle, θ, degree | 42 |
| Critical voltage of helix | 9 |
| unwinding, Vc, V | |
| Spontaneous | 179 |
| polarization, Ps, | |
| nC/cm² | |
| Switching on time, | 70 |
| τ_{ON}, μs | |

### Example 23 FLC Mixture FLC-11-115 Containing One Chiral Guest

The reference mixture.

The FLC mixture has following composition.

| | Content of the component, |
|---|---|
| Chemical formulae | mol. % |
| BPPO-61 | 74.7 |
| 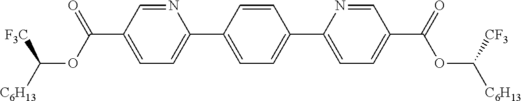 | 25.3 |

Mixture FLC-11-115 shows following properties (at 25° C.):

| | |
|---|---|
| Phase transitions | 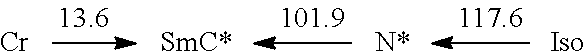 |
| Helix pitch, p0, nm | 124 |
| Tilt angle, θ, degree | 42 |
| Critical voltage of helix | 8.7 |
| unwinding, Vc, V | |
| Spontaneous | 180 |
| polarization, Ps, | |
| nC/cm² | |
| Switching on time, | 42 |
| τ_{ON}, μs | |

### Example 24 FLC Mixture FLC-11-127 Containing One Chiral Guest

The reference mixture.

The FLC mixture has following composition.

| | Content of the component, |
|---|---|
| Chemical formulae | mol. % |
| BPPO-61 | 75 |
| 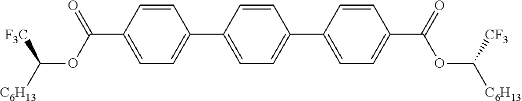 | 25 |

Mixture FLC-11-127 shows following properties (at 25° C.):

| | |
|---|---|
| Phase transitions | 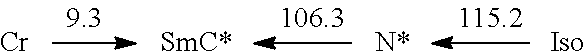 |
| Helix pitch, p₀, nm | 108.7 |
| Tilt angle, θ, degree | 37 |
| Critical voltage of helix | 4.6 |
| unwinding, Vc, V | |
| Spontaneous | 165 |
| polarization, Ps, | |
| nC/cm² | |
| Switching on time, | 60 |
| τ_{ON}, μs | |

It should be understood that the above embodiments are described only for illustrating the present invention, rather than for limiting the present invention. A person skilled in the art may make variations to the above embodiments according to the inventive concept of the present invention.

What is claimed is:

1. A chiral compound for ferroelectric liquid crystal (FLC) material, is of a formula: 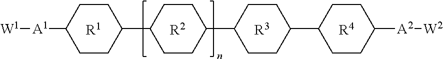
wherein
n is 0 or 1;
R¹, R², R³, and R⁴ each independently are 1,4-phenylene, optionally substituted with halogen or methyl at 2 or 3 positions, or pyrimidine-2,5-diyl, or pyridine-2,5-diyl, provided that at least one of rings R¹ and R⁴ are different from unsubstituted 1,4-phenylene;
A¹ and A² are independently a single C—C bond, O, S, or an ester functional group;
W¹ and W² are chiral groups of shown absolute configuration selected either from a first table below:
first table

| W¹ | W² |
|---|---|
| 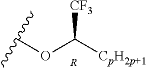 | 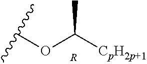 |
| 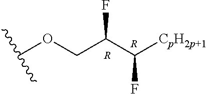 | 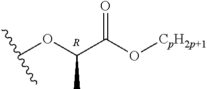 |
| 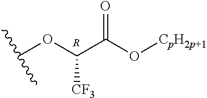 | |
| 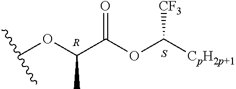 | |
| 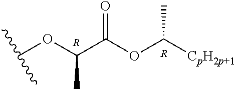 | |
| 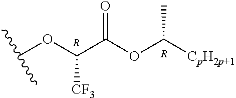 | |
| 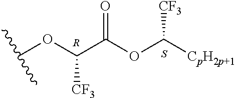 | |
| 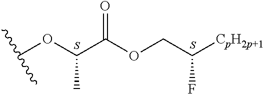 | |
| 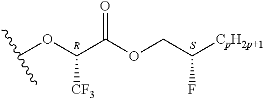 | |
| 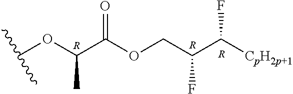 | |
| 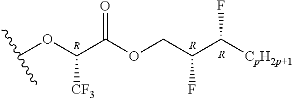 | |

Or W¹ and W² are chiral groups with mirror configuration of each chiral centre shown in a second table below:
second table
W¹ 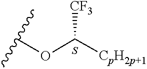 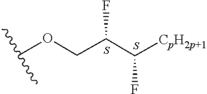 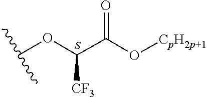 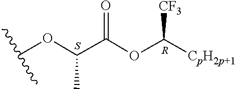 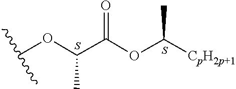 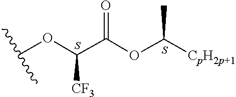 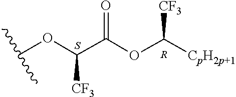 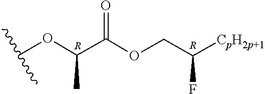 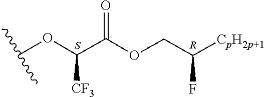 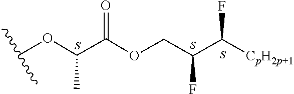 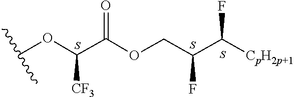
W² 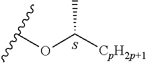 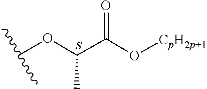
Wherein, CₚH₂ₚ₊₁ is alkyl group, p=2-14, and optionally where one or more CH₂ are independently replaced with CF₂, O, or —CO— groups provided that two O atoms are not linked together.

2. The chiral compound according to claim 1, wherein the chiral compound is S-DFT-TFA6 with a formula below:

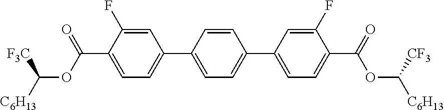

3. The chiral compound according to claim 1, wherein the chiral compound is PDN-TFA6 with a formula below:

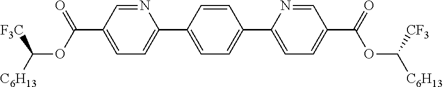

4. A chiral mixture for ferroelectric liquid crystal (FLC) material, comprising at least two chiral compounds of following formulae: 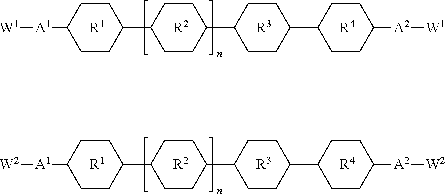
wherein
n is 0 or 1;
R¹, R², R³, and R⁴ each independently are 1,4-phenylene, optionally substituted with halogen or methyl at 2 or 3 positions, or pyrimidine-2,5-diyl, or pyridine-2,5-diyl, provided that at least one of rings R¹ and R⁴ are different from unsubstituted 1,4-phenylene;
A¹ and A² are independently a single C—C bond, O, S, or an ester functional group;
W¹ and W² are chiral groups of shown absolute configuration selected either from a first table below:
first table:
W¹ 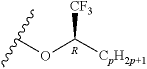 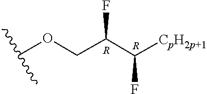 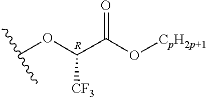 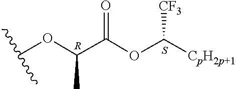 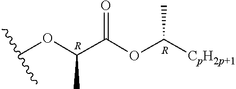 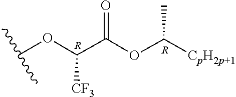 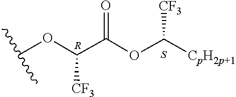 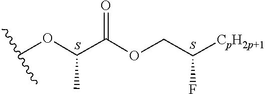 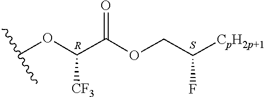 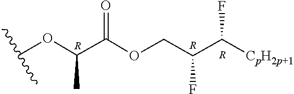 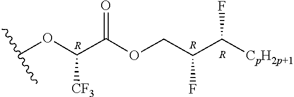
W² 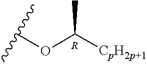 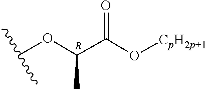
Or chiral groups with mirror configuration of each chiral centre shown in a second table below:
second table:
W¹ 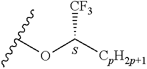 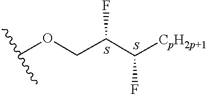 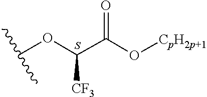 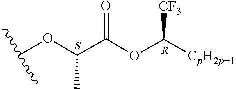 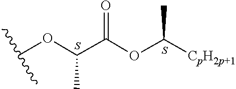 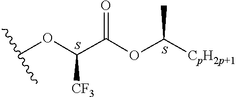 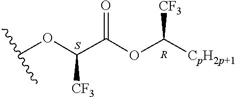 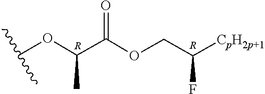 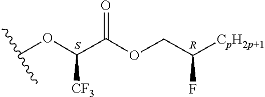 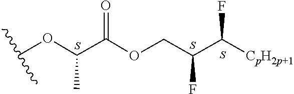 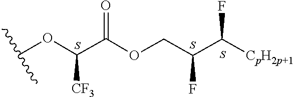
W² 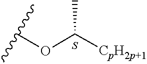 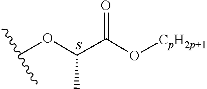
where CₚH₂ₚ₊₁ is alkyl group, p=2-14, and optionally where one or more CH₂ are independently replaced with CF₂, O, or —CO— groups provided that two O atoms are not linked together.

5. The chiral mixture according to claim 4, wherein one of the at least two chiral compounds is PDN-TFA6 with a formula (a):
S-DFT-TFA6 with a first formula (a) below: 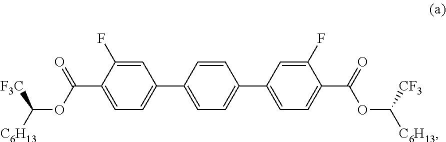
and another of the at least two chiral compounds is Lact-4 with a second formula (b) below:

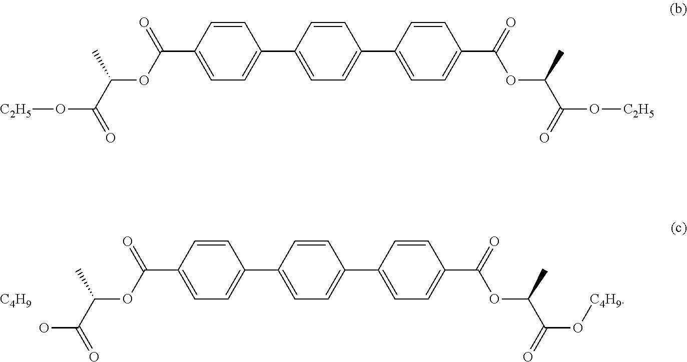

6. The chiral mixture according to claim 5, wherein contents of S-DFT-TFA6 and Lact-4, mol. % in the FLC material are 5%-35%, 2%-15%, respectively.

7. The chiral mixture according to claim 4, wherein one of the at least two chiral compounds is S-PDN-TFA6 with a formula (d): 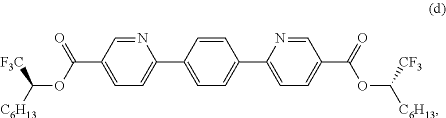
and another of the at least two chiral compounds is Lact-2 with a formula (b) or Lact-4 a formula (c) below:

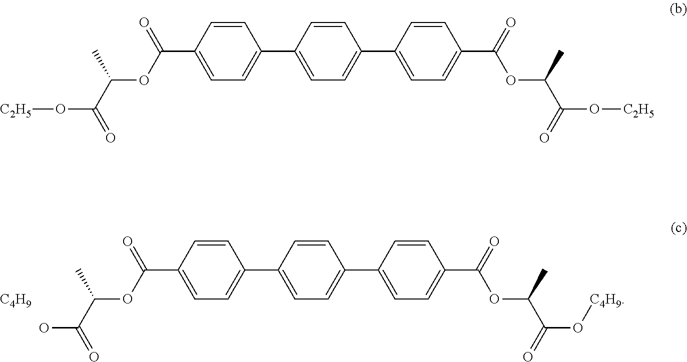

8. The chiral mixture according to claim 7, wherein contents of PDN-TFA6 and Lact-2/Lact-4, mol. % in the FLC material are 5%-35%, 2%-15%, respectively.

9. A ferroelectric liquid crystal (FLC) material in a vertically aligned liquid crystal cell, comprising at least one chiral compound and at least one non-chiral liquid crystal compound, with a helix pitch less than 300 nm, a spontaneous polarization greater than 100 nC/cm², ad a tilt angle greater than 38°.

10. The FLC material according to claim 9, wherein one of the at least one chiral compound is of a formula: 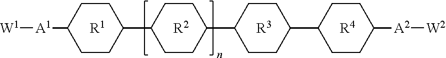
wherein
n is 0 or 1;
R¹, R², R³, and R⁴ each independently are 1,4-phenylene, optionally substituted with halogen or methyl at 2 or 3 positions, or pyrimidine-2,5-diyl, or pyridine-2,5-diyl, provided that at least one of rings R¹ and R⁴ are different from unsubstituted 1,4-phenylene;
A¹ and A² are independently a single C—C bond, O, S, or an ester functional group;
W¹ and W² are chiral groups of shown absolute configuration selected either from a table (i) below:
table (i)
W¹ 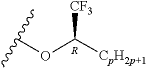 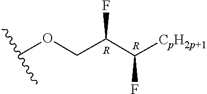 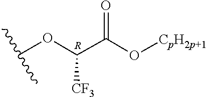 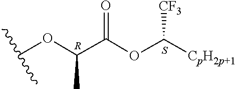 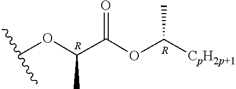 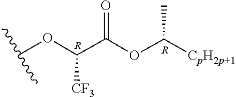 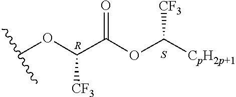 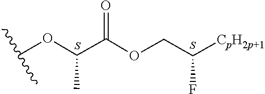 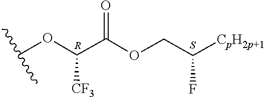 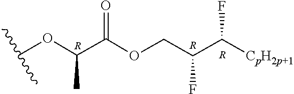 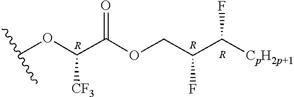
W² 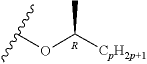 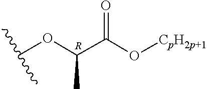
Or W¹ and W² are chiral groups with mirror configuration of each chiral centre shown in a table (ii) below:
table (ii)
W¹ 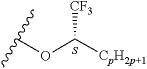 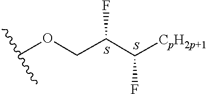 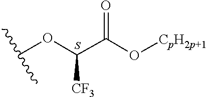 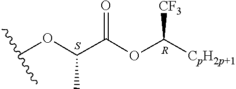 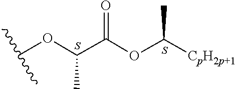 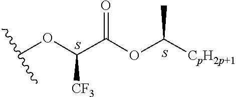 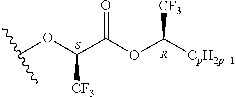 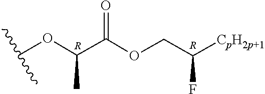 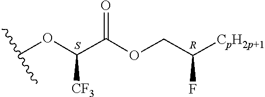 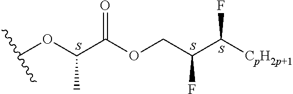 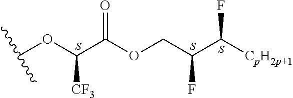
W² 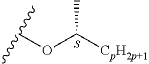 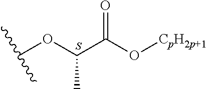
where CₚH₂ₚ₊₁ is alkyl group, p=2-14, and optionally where one or more CH₂ are independently replaced with CF₂, O, or —CO— groups provided that two O atoms are not linked together.

11. The FLC material according to claim 9, wherein one of the at least one chiral compound is S-DFT-TFA-6 with a formula 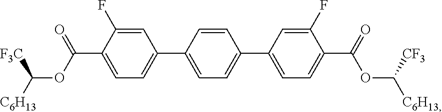
or S-PDN-TFA6 with a formula

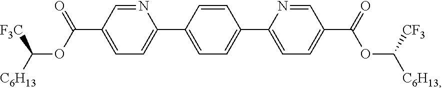

12. The FLC material according to claim 9, wherein the at least one chiral compound comprises two chiral compounds of formulae: 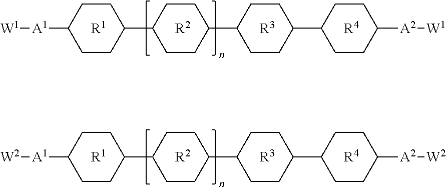
wherein
n is 0 or 1;
R¹, R², R³, and R⁴ each independently are 1,4-phenylene, optionally substituted with halogen or methyl at 2 or 3 positions, or pyrimidine-2,5-diyl, or pyridine-2,5-diyl, provided that at least one of rings R¹ and R⁴ are different from unsubstituted 1,4-phenylene;
A¹ and A² are independently a single C—C bond, O, S, or an ester functional group;
W¹ and W² are chiral groups of shown absolute configuration selected either from a table (iii) below:
table (iii)

| W¹ | | | W² |
|---|---|---|---|
| 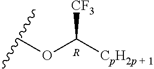 | 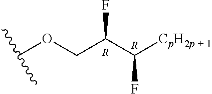 | 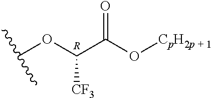 | 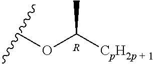 |
| 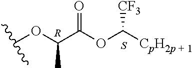 | 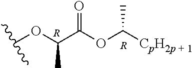 | 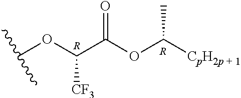 | 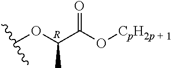 |
| 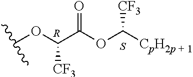 | 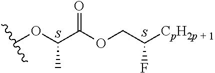 | 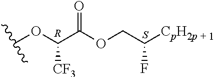 | |
| | |
|---|---|
| 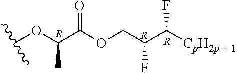 | 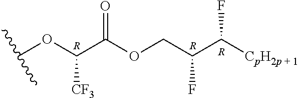 |

Or chiral groups with mirror configuration of each chiral centre shown in a table (iv) below:
table (iv)

| W¹ | | | W² |
|---|---|---|---|
| 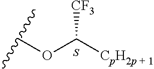 | 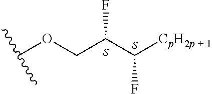 | 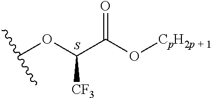 | 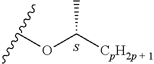 |
| 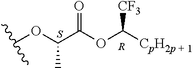 | 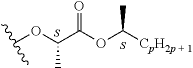 | 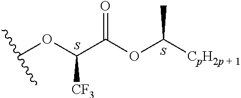 | 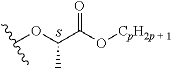 |
| 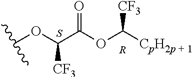 | 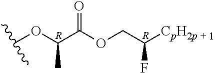 | 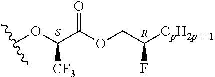 | |
| | |
|---|---|
| 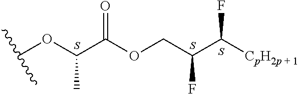 | 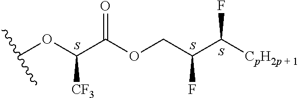 |

where CₚH₂ₚ₊₁ is alkyl group, p=2-14, and optionally where one or more CH₂ are independently replaced with CF₂, O, or —CO— groups provided that two O atoms are not linked together.

13. The FLC material according to claim 9, wherein one of the at least two chiral compounds is below:
S-DFT-TFA6 with a first formula (a) or S-PDN-TFA6 with a formula (b): 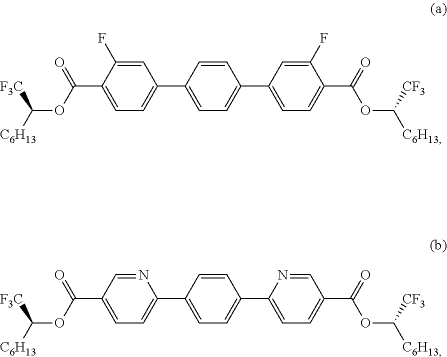
and another of the at least two chiral compounds is Lact-2 with a formula (c) or Lact-4 with a formula (d) below:

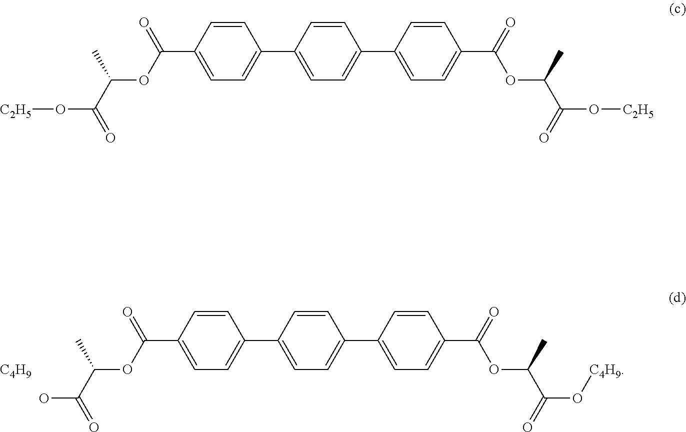

14. The FLC material according to claim 9, wherein the at least one non-chiral liquid crystal compound has smectic C liquid crystal phase and is of formulae: 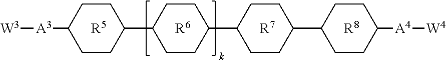
wherein
k is 0 or 1;
R⁵, R⁶, R⁷, and R⁸ independently are 1,4-phenylene, optionally substituted with halogen or methyl at 2 or 3 positions, or pyrimidine-2,5-diyl, or pyridine-2,5-diyl;
A³ and A⁴ are independently a single C—C bond, O, S, or an ester functional group;
W³ and W⁴ are independently alkyl CₘH₂ₘ₊₁ or alkenyl CₘH₂ₘ, where m=4-12, and optionally one or more hydrogens are independently replaced by F, furthermore, optionally one or more CH₂ are independently replaced with CF₂, O, or —CO— groups provided that two O atoms are not linked together.

15. The FLC material according to claim 14, wherein the at least one non-chiral liquid crystal compound comprises BPP-6, wherein BPP-6 consists of following compositions:

| Chemical formula of composition | Content of composition, mol. % |
|---|---|
| 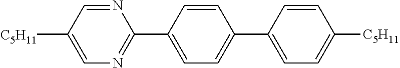 | 4.0 |
| 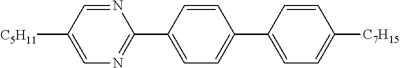 | 5.0 |
| 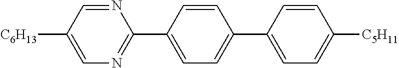 | 49.5 |
| 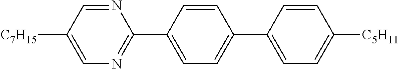 | 19.8 |
| 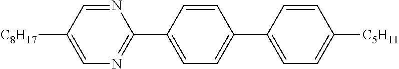 | 2.0 |
| 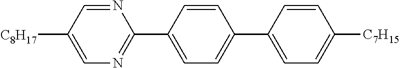 | 19.8 |

16. The FLC material according to claim 14, wherein the at least one non-chiral liquid crystal compound comprises BPP-60, wherein BPP-60 comprises BPPO-61, BPPO-62 and BPPO-63 respectively consisting of following compositions:

| | Content of composition, mol. % | | |
|---|---|---|---|
| Chemical formula of composition | BPPO-61 | BPPO-62 | BPPO-63 |
| 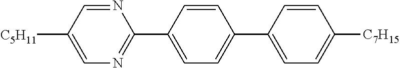 | 14.4 | 12.5 | 10.7 |
| 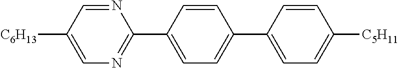 | 32.5 | 28.3 | 24.2 |
| 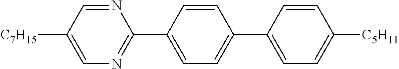 | 14.6 | 12.6 | 10.8 |
| 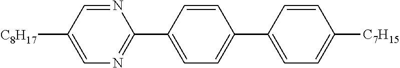 | 19.2 | 16.7 | 14.2 |
| 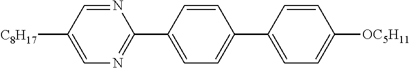 | 10.5 | 16.3 | 21.9 |
| 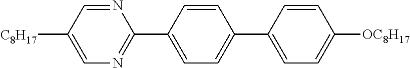 | 8.8 | 13.6 | 18.2 |

17. The FLC material according to claim 14, wherein the at least one non-chiral liquid crystal compound comprises BPP-4, wherein BPP-4 consists of following compositions:

| | Content of composition, |
|---|---|
| Chemical formula of composition | mol. % |
| 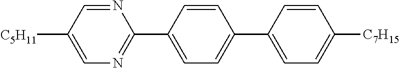 | 17.7 |
| 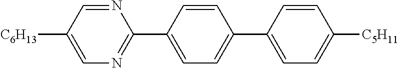 | 40.1 |
| 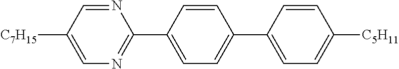 | 18.2 |
| 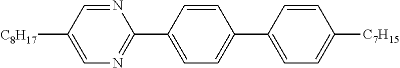 | 24 |

18. The FLC material according to claim 9, wherein the FLC material has a helix pitch less than 300 nm, spontaneous polarization higher than 100 nC/cm², a tilt angle higher than 38 degrees, a Kerr constant greater than 100 nm/V² for c-band of light, a driving voltage less than 2 V/μm for 2π phase modulation of the c-band of the light, and a response time less than 500 μs for the c-band of the light.

19. The FLC material according to claim 9, wherein the FLC material has a helix pitch less than 150 nm, spontaneous polarization higher than 200 nC/cm², a tilt angle higher than 42 degrees, a Kerr constant greater than 300 nm/V² for c-band of light, a driving voltage less than 1 V/μm for 2π phase modulation of the c-band of the light, and a response time less than 100 μs for the c-band of the light.

20. The FLC material according to claim 9, wherein content of the at least one chiral compound is 2%-45%, and content of the at least one non-chiral liquid crystal compound is 55%-85%.
